(19) United States
(12) Patent Application Publication
(10) Pub. No.: US 2026/0070992 A1
(43) Pub. Date: Mar. 12, 2026

(54) ACTRII ANTIBODY FIXED UNIT DOSE TREATMENTS

(71) Applicant: VERSANIS BIO, INC., Indianapolis, IN (US)

(72) Inventor: Lloyd B. KLICKSTEIN, Newton, MA (US)

(21) Appl. No.: 19/105,371

(22) PCT Filed: Aug. 28, 2023
(86) PCT No.: PCT/US2023/072982

§ 371 Date: Feb. 21, 2025

Related U.S. Application Data
(60) Provisional application No. 63/373,684, filed on Aug. 26, 2022.
(60) Provisional application No. 63/378,128, filed on Oct. 3, 2022.

(51) Int. Cl. C07K 16/28; A61K 39/00; A61K 45/06; A61P 3/00

(52) U.S. Cl. CPC C07K 16/2863; A61K 45/06; A61P 3/00; A61K 2039/505; A61K 2039/545; C07K 2317/565

(57) ABSTRACT

Provided herein are compositions and methods for subcutaneous administration of a fixed unit dose of an ActRII antibody for disease treatment, wherein the treatment is administered across a range of subject body weights. As provided herein, the ActRII antibody fixed unit dose may include about 25 mg to about 600 mg, e.g., about 150 mg of an ActRII antibody, e.g., in a volume of about 1 ml.

### CROSS-REFERENCE TO RELATED APPLICATIONS

This application claims priority to U.S. Provisional Patent Application Nos. 63/373,684 filed Aug. 26, 2022, and 63/378,128 filed Oct. 3, 2022, the contents of which are incorporated herein by reference in their entirety.

### INCORPORATION BY REFERENCE OF SEQUENCE LISTING

The contents of the electronic sequence listing (VRNS_010_02WO_SeqList_ST26.xml; Size: 4,428,735 bytes; and Date of Creation: Aug. 25, 2023) are herein incorporated by reference in their entirety.

### BACKGROUND

Therapeutic antibodies are often administered based on a subject's body weight. Such customization however requires a medical professional to plan and administer the dose, among other things. An attractive alternative is to administer a fixed dose, which can be administered to subjects across a range of body weights. There is an added advantage that a fixed dose, could even be self-administered at home by the subject, for example using a hand-held subcutaneous injector. For therapeutic antibodies, fixed dosing however requires pharmacokinetic (PK) and pharmacodynamic (PD) optimization to achieve a desired range of exposure, while taking into account antigen affinity, antigen density, phagocytic clearance, IgG recycling, errors associated with erroneous self-administration, and the consequences of a missed dose. Lack of such PK/PD optimization would lead to ineffective dosing and would interfere with the efficacy of an otherwise life-saving treatment.

Treatments with ActRII antibodies are useful for a variety of diseases such as metabolic diseases, muscle wasting diseases, heart diseases, and liver diseases. Currently the clinical use of such antibodies does not include a fixed dosing paradigm. Thus, a need exists for PK/PD optimized fixed unit doses of ActRII antibodies, applicable across a range of body weights, and useful for self-administration, and accordingly are provided herein.

### SUMMARY

Provided herein are compositions and methods for subcutaneous administration of fixed unit doses of an ActRII antibody, which may be used to treat a variety of diseases, and wherein the fixed unit dose is administered to subjects with a range of body weights. In exemplary embodiments, a pharmaceutical composition in a fixed unit dose form comprises a dose of about 25 mg to about 600 mg of an ActRII antibody. In some exemplary embodiments, a pharmaceutical composition in a fixed unit dose form comprises a dose of about 100 to about 400 mg of an ActRII antibody. In some exemplary embodiments, the fixed unit dose comprises a dose of about 150 mg of an ActRII antibody. In some exemplary embodiments, the fixed unit dose comprises a dose of about 300 mg of an ActRII antibody.

In some embodiments, the ActRII antibody comprises the CDR amino acid sequences of SEQ ID NOS: 1-6. In some embodiments, the ActRII antibody comprises the VH amino acid sequence of SEQ ID NO: 7, or a sequence with at least 80% sequence identity thereto, and the VL amino acid sequence of SEQ ID NO: 8, or a sequence with at least 80% sequence identity thereto.

In some embodiments, the ActRII antibody comprises the amino acid sequence of SEQ ID NO: 9, or a sequence with at least 80% sequence identity thereto, and the amino acid sequence of SEQ ID NO: 10, or a sequence with at least 80% sequence identity thereto.

In some embodiments, the ActRII antibody comprises one or more of the CDR amino acid sequences of SEQ ID NOS: 11-75. In some embodiments, the ActRII antibody comprises one or more of the CDR amino acid sequences of SEQ ID NOS: 76-81. In some embodiments, the ActRII antibody comprises one or more of the CDR amino acid sequences of SEQ ID NOS: 82-5049.

In some embodiments, the ActRII antibody is specific for ActRIIA and/or ActRIIB.

In some embodiments, the ActRII antibody is present in the fixed unit dose in a concentration of about 25 mg/ml, about 50 mg/ml, about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, about 300 mg/ml, about 325 mg/ml, about 350 mg/ml, about 375 mg/ml, about 400 mg/ml, about 425 mg/ml, about 450 mg/ml, about 475 mg/ml, about 500 mg/ml, about 525 mg/ml, about 550 mg/ml, about 575 mg/ml, or at about 600 mg/ml. In some embodiments, the ActRII antibody is present in the fixed unit dose in a concentration of about 150 mg/ml.

In some embodiments, the fixed unit dose is a volume of about 0.25 ml, about 0.5 ml, about 0.75 ml, about 1.0 ml, about 1.24 ml, about 1.5 ml, about 1.75 ml, about 2.0 ml, about 2.25 ml, about 2.5 ml, about 2.75 ml, about 3.0 ml, about 3.25 ml, about 3.5 ml, about 3.75 ml, about 4.0 ml, about 4.25 ml, about 4.5 ml, about 4.75 ml, or about 5.0 ml. In some embodiments, the fixed unit dose is a volume of about 1 ml. In some embodiments, the fixed unit dose is a volume of about 2 ml. In some embodiments, the composition is housed in an injector. In some embodiments, the injector is a needle or a syringe.

In some embodiments, the pharmaceutical composition comprises a hormone agonist and the ActRII antibody. In some embodiments, the hormone agonist is an incretin agonist. In some embodiments, the incretin agonist is selected from the group consisting of exenatide, exenatide extended-release, dulaglutide, liraglutide, lixisenatide, semaglutide, tirzepatide, cotadutide, noiiglutide, oxyntomodulin, retatrutide, albiglutide, beinaglutide, PEG-loxenatide, pemvidutide, and danuglipron.

In some embodiments, the hormone agonist is selected from the group consisting of long-acting amylin receptor agonists, dual amylin calcitonin receptor agonists (DACRAs), and Peptide YY agonists.

In some embodiments, provided herein is a method of treating a disease in a subject in need thereof, comprising subcutaneously administering to the subject a fixed unit dose of an ActRII antibody. In some embodiments, the ActRII antibody is present in at about 25 mg to about 600 mg per fixed unit dose. In some embodiments, the ActRII antibody is present in at about 100 mg to about 400 mg per fixed unit dose. In some embodiments, the ActRII antibody is present at about 150 mg per fixed unit dose. In some embodiments, the ActRII antibody is present at about 300 mg per fixed unit dose.

In some embodiments, the ActRII antibody fixed unit dose is administered to the subject about every day, about six times a week, about five times a week, about four times a week, about three times a week, about twice a week, about once every week, about once every two weeks, about every three weeks, about every four weeks, about every five weeks, about every six weeks, about every seven weeks, or about every eight weeks. In some embodiments, the ActRII antibody fixed unit dose is administered to the subject about once every week.

In some embodiments, administration of an ActRII antibody loading dose precedes administration of the ActRII antibody fixed unit dose. In some embodiments, the ActRII antibody loading dose is administered intravenously at about 3 mg/kg to about 50 mg/kg.

In some embodiments, the ActRII antibody loading dose is administered intravenously at about 10 mg/kg. In some embodiments, the ActRII antibody loading dose is administered intravenously at about 30 mg/kg.

In some embodiments, the ActRII antibody loading dose is administered in a dose of about 150 mg to about 4500 mg. In some embodiments, the ActRII antibody loading dose is administered in a dose of about 210 mg. In some embodiments, the ActRII antibody loading dose is administered in a dose of about 700 mg.

In some embodiments, the ActRII antibody loading dose is administered in a dose of about 25 mg to about 600 mg. In some embodiments, the ActRII antibody loading dose is administered in a dose of about 100 mg to about 400 mg. In some embodiments, the ActRII antibody loading dose is administered in a dose of about 150 mg. In some embodiments, the ActRII antibody loading dose is administered in a dose of about 300 mg. In some embodiments, the ActRII antibody loading dose is administered intravenously. In other embodiments, the ActRII antibody loading dose is administered subcutaneously.

In some embodiments, the ActRII antibody loading dose is administered about 1 day, about 2 days, about 3 days, about 4 days, about 5 days, about 6 days, about 1 week, about 2 weeks, about 3 weeks, about 4 weeks, or about 5 weeks before the administration of the ActRII antibody fixed unit dose.

In some embodiments, the subject's disease is a metabolic disease. In some embodiments, the metabolic disease is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

In some embodiments, the monogenetic disorder associated with obesity is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

In some embodiments, the metabolic disease is Prader-Willi syndrome.

In some embodiments, the subject's disease is a muscle wasting disease or sarcopenia. In some embodiments, the subject's disease is aging related muscle dysfunction. In some embodiments, the subject's disease is heart disease. In some embodiments, the subject's disease is liver disease.

In some embodiments, the subject has a body mass index (BMI) of 30 or greater. The method of any one of claims 20-43, wherein the subject has a body mass index (BMI) of 27 or greater and has one or more obesity-related disorders. In some embodiments, the subject weighs about 200 kg or less. In some embodiments, the subject weighs more than about 200 kg.

In some embodiments, the ActRII antibody used in the method of treatment comprises the CDR amino acid sequences of SEQ ID NOS: 1-6. In some embodiments, the ActRII antibody comprises the VH amino acid sequence of SEQ ID NO: 7, or a sequence with at least 80% sequence identity thereto, and the VL amino acid sequence of SEQ ID NO: 8, or a sequence with at least 80% sequence identity thereto. In some embodiments, the ActRII antibody comprises the HC amino acid sequence of SEQ ID NO: 9, or a sequence with at least 80% sequence identity thereto, and the LC amino acid sequence of SEQ ID NO: 10, or a sequence with at least 80% sequence identity thereto.

In some embodiments, the ActRII antibody used in the method of treatment comprises the CDR amino acid sequences of one or more of SEQ ID NOS: 11-75. In some embodiments, the ActRII antibody used in the method of treatment comprises the CDR amino acid sequences of one or more of SEQ ID NOS: 76-81. In some embodiments, the ActRII antibody used in the method of treatment comprises the CDR amino acid sequences of one or more of SEQ ID NOS: 82-5049.

In some embodiments, the ActRII antibody is specific for ActRIIA and/or ActRIIB.

In some embodiments, the ActRII antibody is present in the fixed unit dose in a concentration of about 25 mg/ml, about 50 mg/ml, about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, about 300 mg/ml, about 325 mg/ml, about 350 mg/ml, about 375 mg/ml, about 400 mg/ml, about 425 mg/ml, about 450 mg/ml, about 475 mg/ml, about 500 mg/ml, about 525 mg/ml, about 550 mg/ml, about 575 mg/ml, or at about 600 mg/ml.

In some embodiments, the fixed unit dose is a volume of about 0.25 ml, about 0.5 ml, about 0.75 ml, about 1.0 ml, about 1.24 ml, about 1.5 ml, about 1.75 ml, about 2.0 ml, about 2.25 ml, about 2.5 ml, about 2.75 ml, about 3.0 ml, about 3.25 ml, about 3.5 ml, about 3.75 ml, about 4.0 ml, about 4.25 ml, about 4.5 ml, about 4.75 ml, or about 5.0 ml.

In some embodiments, the fixed unit dose is housed in an injector configured for subcutaneous administration. In some embodiments, the injector is a needle or a syringe. In some embodiments, the fixed unit dose is designed to be self-administered.

In some embodiments, a hormone agonist is administered as a part of a combination therapy with the ActRII antibodies of the disclosure.

In some embodiments, the hormone agonist is an incretin agonist. In some embodiments, the incretin agonist is selected from the group consisting of exenatide, exenatide extended-release, dulaglutide, liraglutide, lixisenatide, semaglutide, tirzepatide, cotadutide, noiiglutide, oxyntomodulin, retatrutide, albiglutide, beinaglutide, PEG-loxenatide, pemvidutide, and danuglipron. In some embodiments, the hormone agonist is selected from the group consisting of long-acting amylin receptor agonists, dual amylin calcitonin receptor agonists (DACRAs), and Peptide YY agonists.

In some embodiments, the hormone agonist is administered prior to the ActRII antibody. In some embodiments, the ActRII antibody is administered prior to the hormone agonist. In some embodiments, the ActRII antibody and the hormone agonist are co-formulated to be administered as a combination therapy.

### BRIEF DESCRIPTION OF THE DRAWINGS

FIG. 1 is a schematic of an exemplary clinical study design that was implemented to evaluate the pharmacodynamics and pharmacokinetics of subcutaneous dosing of an ActRII antibody (BYM338, also known as bimagrumab).

FIG. 2 is a table of the demographics of the subjects, for the clinical study shown in FIG. 1.

FIG. 3 is a table of the physical characteristics of the subjects, prior to starting the study shown in FIG. 1.

FIG. 4 is a table of summarized PK parameters Cmax and Cmin from the study shown in FIG. 1.

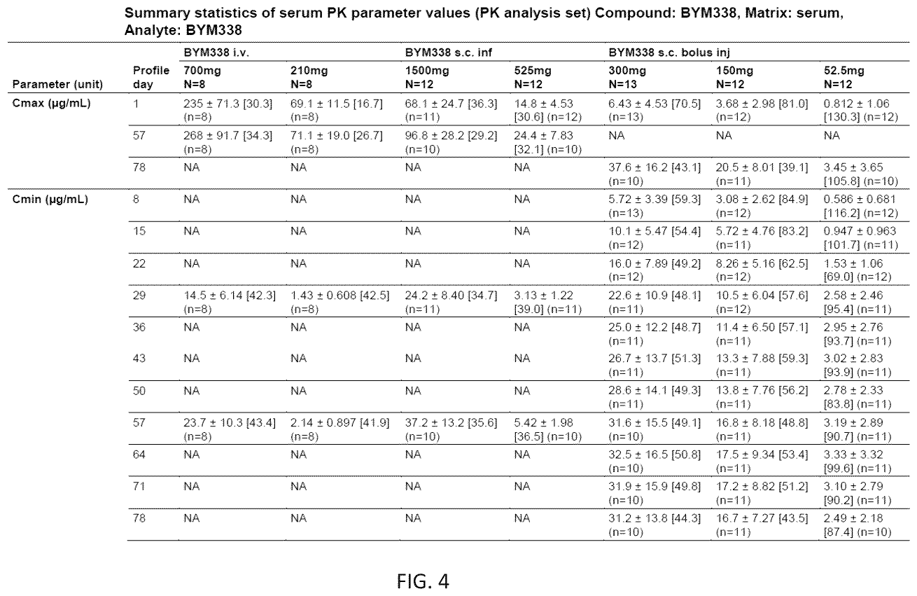

FIG. 5 is a table summarizing PK parameters including Tmax and AUC from the study shown in FIG. 1.

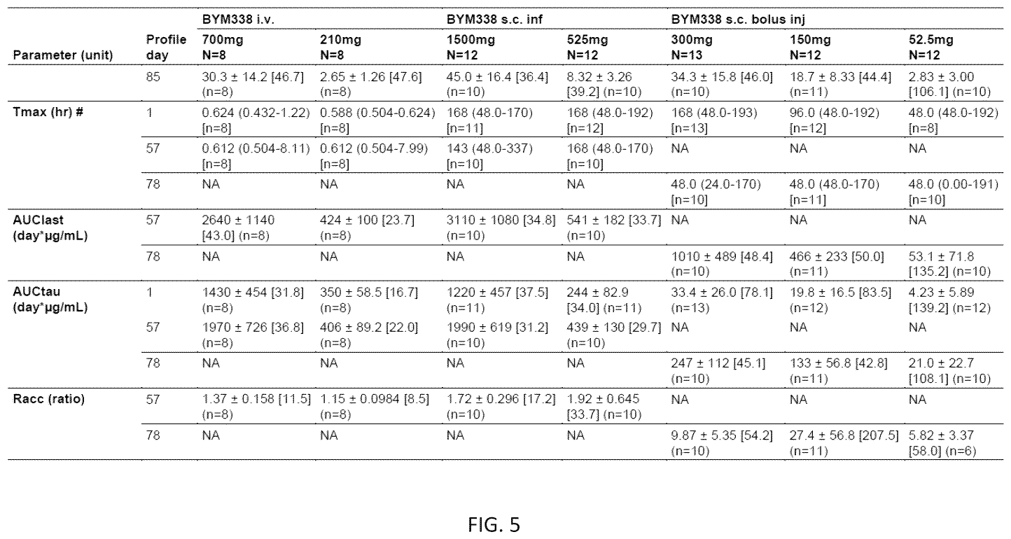

FIG. 6 is a semi-log graph of model fit mean serum concentration profiles for intravenous administration of bimagrumab at 210 mg and 700 mg concentrations, from the study shown in FIG. 1.

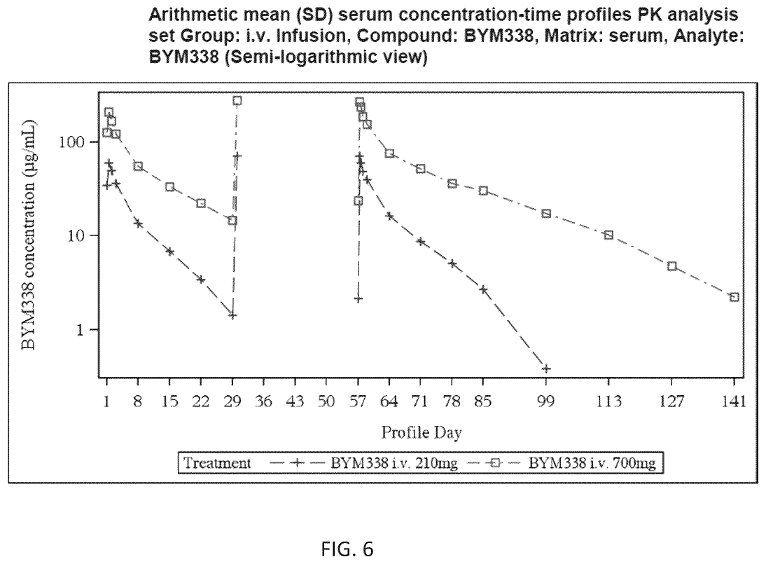

FIG. 7 is a semi-log graph of model fit mean serum concentration profiles for subcutaneous infusion administration of bimagrumab at 1500 mg and 525 mg concentrations, from the study shown in FIG. 1.

FIG. 8 is a semi-log graph of model fit mean serum concentration profiles for subcutaneous bolus administration of bimagrumab at 150 mg, 300 mg, and 52.5 mg concentrations, from the study shown in FIG. 1.

FIG. 9 is a table summarizing the lean body mass and fat body mass results from the study shown in FIG. 1.

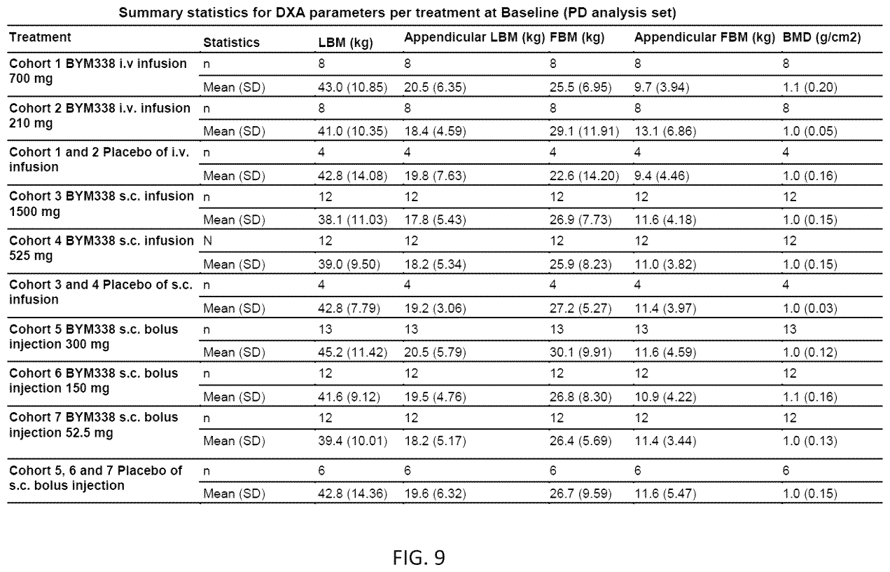

FIG. 10 is a line graph of the lean body mass results of intravenous infusion from the study shown in FIG. 1.

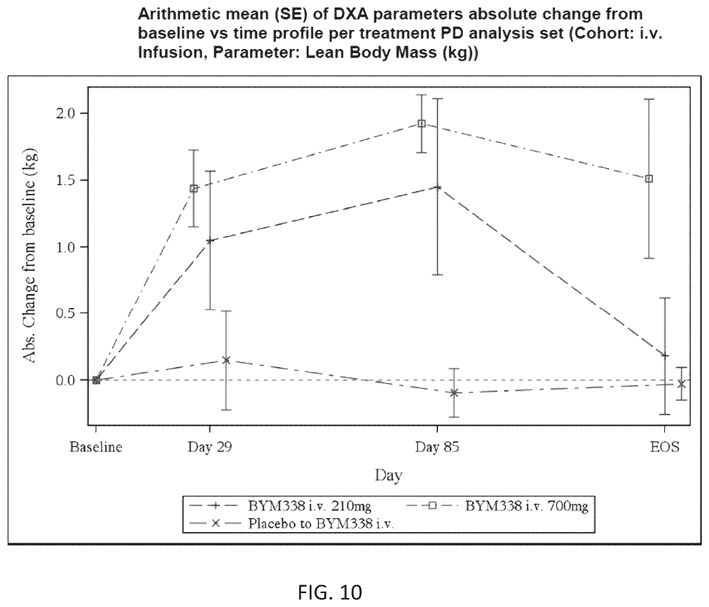

FIG. 11 is a line graph of the appendicular lean body mass results of intravenous infusion from the study shown in FIG. 1.

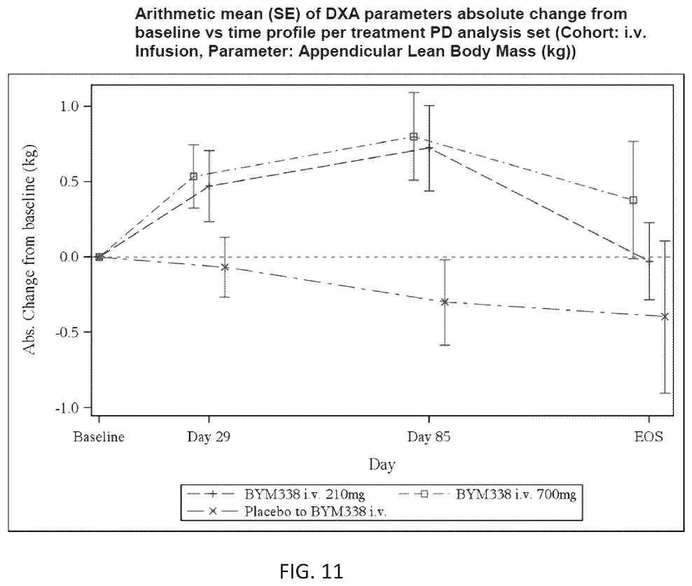

FIG. 12 is a line graph of the lean body mass results of subcutaneous infusion from the study shown in FIG. 1.

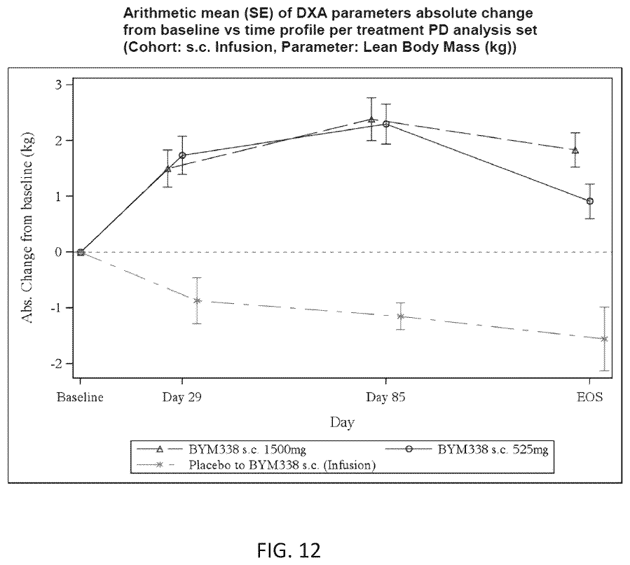

FIG. 13 is a line graph of the appendicular lean body mass results of subcutaneous infusion from the study shown in FIG. 1.

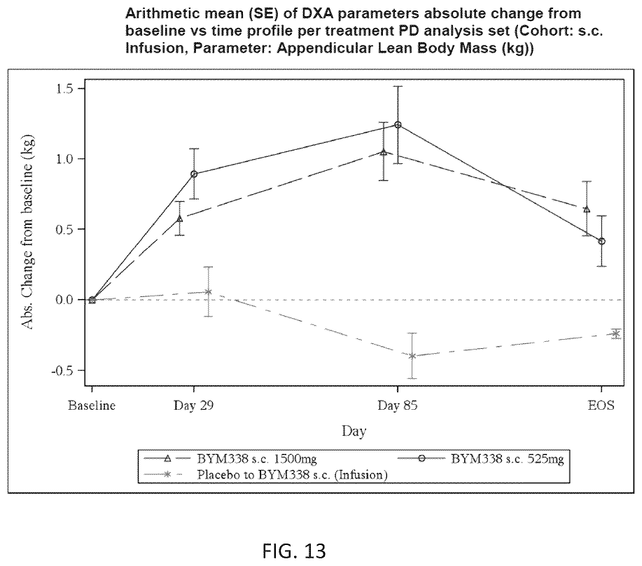

FIG. 14 is a line graph of the lean body mass results of subcutaneous bolus administration from the study shown in FIG. 1.

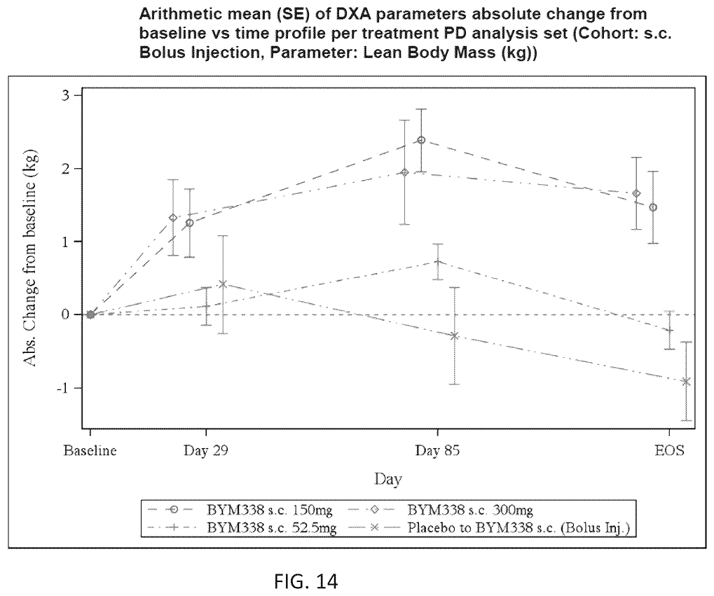

FIG. 15 is a line graph of the appendicular lean body mass results of subcutaneous bolus administration from the study shown in FIG. 1.

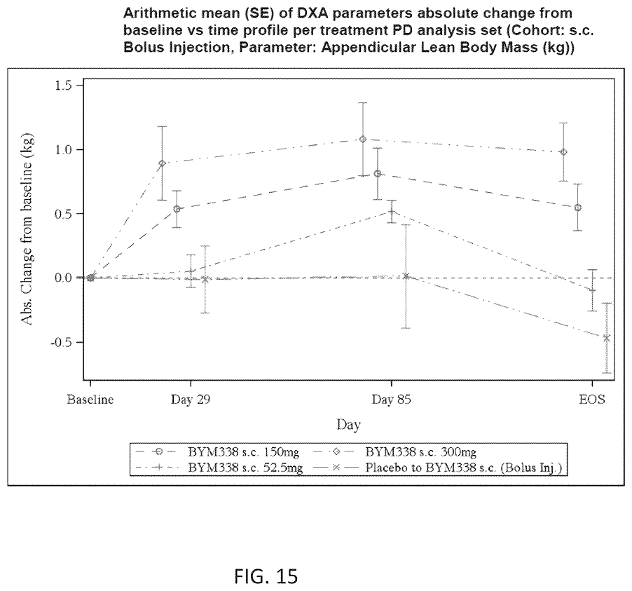

FIG. 16 is a line graph of the fat body mass results of intravenous infusion from the study shown in FIG. 1.

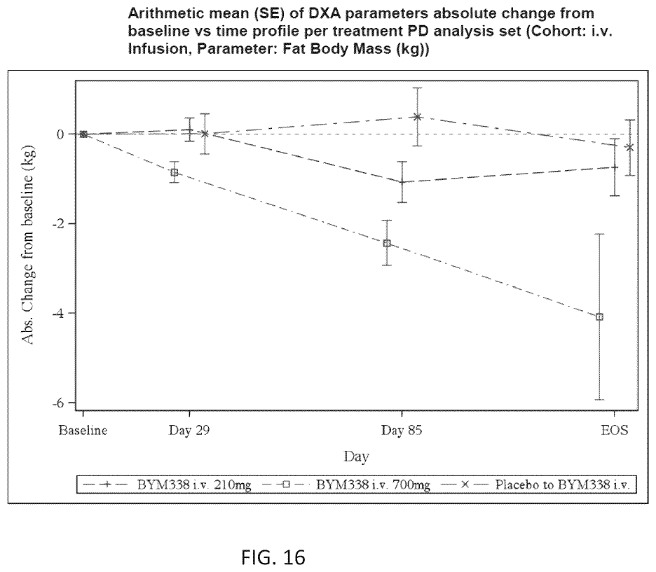

FIG. 17 is a line graph of the fat body mass results of subcutaneous infusion from the study shown in FIG. 1.

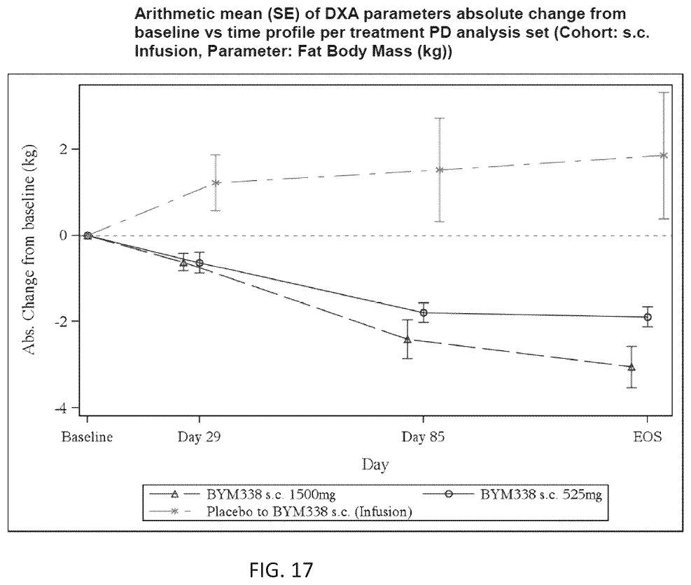

FIG. 18 is a line graph of the fat body mass results of subcutaneous bolus administration from the study shown in FIG. 1.

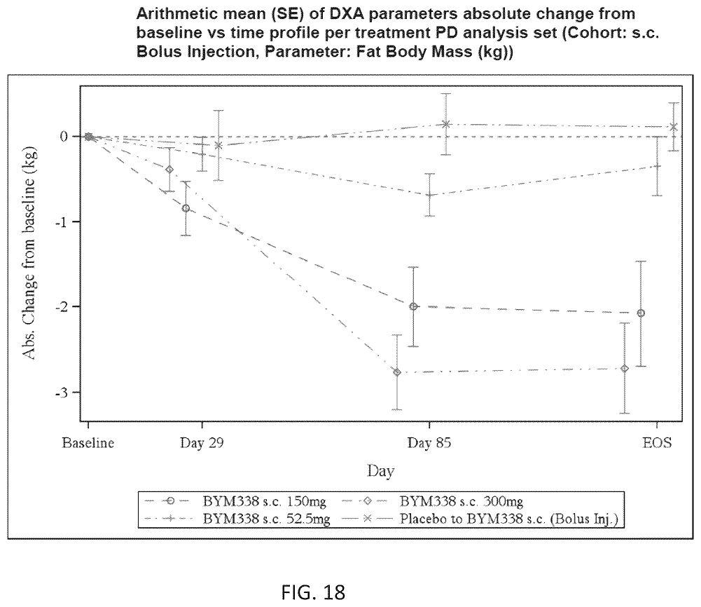

FIG. 19 is a line graph of model-predicted receptor occupancy (RO) of bimagrumab and associated dosing effects on fat body mass (FBM) loss and lean body mass (LBM) increase.

FIG. 20 is a line graph and table of model predicted time to reach various bimagrumab concentrations in a population of 2500 subjects, each weighing 100 kg, in which 300 mg bimagrumab is administered weekly and subcutaneously. Threshold levels of 5 μg/ml and 10 μg/ml are indicated in the graph and in the table below.

FIG. 21 is a line graph and table of model-predicted time to reach various bimagrumab concentrations in a population of 2500 subjects, of weights from 60 to 140 kg, in which 300 mg bimagrumab is administered weekly and subcutaneously. The time to threshold levels of 5 μg/ml and 10 μg/ml are indicated in the table below.

FIG. 22 is a line graph of model-predicted bimagrumab concentration over time in a population of subjects, of weights from 50 to 200 kg, in which 300 mg bimagrumab is administered weekly and subcutaneously without a loading dose on day 1.

FIG. 23 is a line graph of model-predicted change in total fat body mass over time in a population of subjects, of weights from 50 to 200 kg, in which 300 mg bimagrumab is administered weekly and subcutaneously without a loading dose on day 1.

FIG. 24 is a line graph of model-predicted bimagrumab concentration over time in a population of subjects, of weights from 50 to 200 kg, in which 300 mg bimagrumab is administered weekly and subcutaneously with two doses of 300 mg bimagrumab administered subcutaneously on day 1.

FIG. 25 is a line graph of model-predicted change in total fat body mass over time in a population of subjects, of weights from 50 to 200 kg, in which 300 mg bimagrumab is administered weekly and subcutaneously with two doses of 300 mg bimagrumab administered subcutaneously on day 1.

FIG. 26 is a line graph of model-predicted bimagrumab concentration over time in a population of subjects, of weights from 50 to 200 kg, in which 300 mg bimagrumab is administered weekly and subcutaneously with three doses of 300 mg bimagrumab administered subcutaneously on day 1.

FIG. 27 is a line graph of model predicted change in total fat body mass over time in a population of subjects, of weights from 50 to 200 kg, in which 300 mg bimagrumab is administered weekly and subcutaneously with three doses of 300 mg bimagrumab administered subcutaneously on day 1.

FIG. 28 is a line graph of a model predicted comparison of the effect of loading doses (0, 1, or 2 loading doses) on bimagrumab concentration over time in a population of subjects weighing 100 kg in which in which 300 mg bimagrumab is administered weekly and subcutaneously.

FIG. 29 is a line graph of a model predicted comparison of the effect of loading doses (0, 1, or 2 loading doses) on the change in total fat body mass overtime for a population of subjects with a weight of 100 kg in which in which 300 mg bimagrumab is administered weekly and subcutaneously.

FIG. 30 is a line graph of model-predicted bimagrumab concentration over time in a population of subjects, of weights from 50 to 200 kg, in which 600 mg bimagrumab is administered weekly and subcutaneously without a loading dose on day 1.

FIG. 31 is a line graph of model-predicted change in total fat body mass over time in a population of subjects, of weights from 50 to 200 kg, in which 600 mg bimagrumab is administered weekly and subcutaneously without a loading dose on day 1.

FIG. 32 is a line graph of the model-predicted effect of bimagrumab concentration on fat body mass and lean body mass in a population of subjects with individual variability of response.

FIG. 33 is a line graph of model-predicted bimagrumab concentration over time in a population of subjects with individual variability of response. Subject weights range from 50 to 200 kg and 300 mg bimagrumab is administered weekly and subcutaneously without a loading dose on day 1.

### DETAILED DESCRIPTION

### I. Definitions

Unless otherwise defined herein, scientific and technical terms used herein shall have the meanings that are commonly understood by those of ordinary skill in the art. Generally, nomenclature used in connection with, and techniques of, chemistry, molecular biology, cell biology, immunology, pharmacology, and protein chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “an agent” refers to one or mixtures of such candidates, and reference to “a method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the terms “identity” and “identical,” when referring to a comparison of two sequences, refers to the percentage of exact matching residues in an alignment of a sequence provided herein to a reference sequence, such as an alignment generated by a BLAST algorithm or other alignment algorithms known in the art. Identity may be calculated based on an alignment of a full-length sequence provided herein and a full-length reference sequence. Identity may also be calculated based on a partial alignment of a sequence provided herein and a reference sequence, if the reference sequence is longer than a sequence provided herein. Identity may also be calculated based on a partial alignment of a sequence provided herein and a reference sequence, if the reference sequence is shorter than a sequence provided herein. Thus, when aligning two sequences, according to the aforementioned, a query sequence “shares at least x % identity to” a subject sequence if in the alignment of the two sequences, at least x % (rounded down) of the residues in the subject sequence are aligned as an exact match to a corresponding residue in the query sequence, wherein the numerator is the number of exact matches and the denominator is the length of the query sequence. In some embodiments, the denominator may alternatively be the length of the query sequence minus any gaps of two or more non-matching residues. Where the subject sequence has variable positions (e.g., residues denoted X), an alignment to any residue in the query sequence is counted as a match.

As used herein, “antibody” includes reference to an immunoglobulin molecule immunologically reactive with a particular antigen, including both polyclonal and monoclonal antibodies. The term includes humanized antibodies, chimeric antibodies e.g., murine variable region with a human constant region) and conjugated antibodies. The term “antibody” also includes antigen binding forms of antibodies, including fragments that retain antigen-binding capability (e.g., Fab′, F(ab′)₂, Fab, single chain variable fragments (scFv) containing VH and VL sequences linked together in one chain, and single chain antibody fragments (scAb). The term antibody also includes bivalent or bispecific molecules, diabodies, triabodies, and tetrabodies.

The terms “treatment”, “treating” and the like are used herein to generally mean obtaining a desired pharmacologic and/or physiologic effect with a therapeutic agent. The effect may be prophylactic in terms of completely or partially preventing a disease or symptom thereof, e.g., reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject's treatment will be administered during the symptomatic stage of the disease, and in some embodiments, after the symptomatic stage of the disease.

The terms “disease” and “disorder” may be used interchangeably to describe any affliction affecting the subject and for which there is one or more symptoms.

The term “dose,” as used herein, refers to the amount of therapeutic agent administered to a subject to achieve a therapeutic objective.

The term “fixed dose” as used herein, refers to a dose that is not individualized to a particular subject. The fixed dose may be applicable to a population of subjects with a range of body weights.

The term “loading dose” refers to one or more doses of a therapeutic agent that are administered in addition to a treatment comprising a periodic administration of a fixed unit dose. As used herein a “loading dose” may refer to one or more doses of a therapeutic agent which are the same concentration, a lower concentration, or a higher concentration than the fixed unit dose. In some embodiments, a loading dose is administered prior to the start of a treatment comprising a periodic administration of a fixed unit dose. In some embodiments, a loading dose is administered simultaneous with the start of a treatment comprising a periodic administration of a fixed unit dose.

The terms “individual,” “subject,” and “patient” are used interchangeably herein and refer to any subject for whom treatment is desired. The subject may be a mammalian subject. Mammalian subjects include, e. g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

### II. ActRII Antibodies

The disclosure provides compositions and methods for subcutaneous administration of ActRII antibodies at fixed unit doses, which may be used to treat a range of diseases, and wherein the fixed unit dose is administered to subjects with a range of body weights. In exemplary embodiments, an ActRII antibody fixed unit dose comprises about 25 mg to about 600 mg of an ActRII antibody administered subcutaneously. In some exemplary embodiments, an ActRII antibody fixed unit dose comprises about 100 mg to about 400 mg of an ActRII antibody administered subcutaneously. In some exemplary embodiments, an ActRII antibody fixed unit dose comprises about 300 mg of an ActRII antibody administered subcutaneously. In some exemplary embodiments, an ActRII antibody fixed unit dose comprises about 150 mg of an ActRII antibody administered subcutaneously. As provided herein, treatments comprising periodic administration of an ActRII antibody fixed unit dose may be used to treat diseases, including but not limited to: metabolic diseases, muscle wasting diseases, heart diseases, and liver diseases.

The fixed unit doses provided herein comprise ActRII antibodies that bind to the activin receptors ActRIIA and/or ActRIIB. The administration of ActRII antibodies has been previously shown to not only increase lean muscle mass, but also to decrease fat mass and improve glycemic control in clinical studies (WO2010125003A1, WO2018116201A1, and WO2021044287A1 the contents of which are incorporated in their entirety, and Heymsfield et al. 2021; 4 (1):e2033457, JAMA).

### Exemplary Antibodies

In some embodiments, exemplary antibodies of the disclosure comprise the sequence of bimagrumab (BYM338). The below table provides the relevant complementary determining region (CDR), variable heavy chain (VH), variable light chain (VL), heavy chain (HC), and light chain (LC) amino acid sequences for bimagrumab. In some embodiments, exemplary antibodies of the disclosure comprise the sequences related to bimagrumab (BYM338).

Bimagrumab Heavy Chain Complementarity
Determining Regions (CDRs)
CDRH1-
(SEQ ID NO: 1)
GYTFTSSYIN
CDRH2-
(SEQ ID NO: 2)
TINPVSGSTSYAQKFQ
CDRH3-
(SEQ ID NO: 3)
GGWFDY
Bimagrumab Light Chain Complementarity
Determining Regions (CDRs)
CDRL1-
(SEQ ID NO: 4)
TGTSSDVGSYNYVN
CDRL2-
(SEQ ID NO: 5)
MIYGVSKRPS
CDRL3-
(SEQ ID NO: 6)
GTFAGGSYYG
Bimagrumab Variable Heavy Chain (VH)
(SEQ ID NO: 7)
QVQLVQSGAEVKKPGASVKVSCKASGYTFTSSYINWVRQAPGQGLEW
MGTINPVSGSTSYAQKFQGRVTMTRDTSISTAYMELSRLRSDDTAVY
YCARGGWFDYWGQGTLVTVSS
Bimagrumab Variable Light Chain (VL)
(SEQ ID NO: 8)
QSALTQPASVSGSPGQSITISCTGTSSDVGSYNYVNWYQQHPGKAPK
LMIYGVSKRPSGVSNRFSGSKSGNTASLTISGLQAEDEADYYCGTFA
GGSYYGVFGGGTKLTVLGQ
Bimagrumab Heavy Chain (HC)
(SEQ ID NO: 9)
QVQLVQSGAEVKKPGASVKVSCKASGYTFTSSYINWVRQAPGQGLEW
MGTINPVSGSTSYAQKFQGRVTMTRDTSISTAYMELSRLRSDDTAVY
YCARGGWFDYWGQGTLVTVSSASTKGPSVFPLAPSSKSTSGGTAALG
CLVKDYFPEPVTVSWNSGALTSGVHTFPAVLQSSGLYSLSSVVTVPS
SSLGTQTYICNVNHKPSNTKVDKRVEPKSCDKTHTCPPCPAPEAAGG
PSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAP
IEKTISKAKGQPREPQVYTLPPSREEMTKNQVSLTCLVKGFYPSDIA
VEWESNGQPENNYKTTPPVLDSDGSFFLYSKLTVDKSRWQQGNVFSC
SVMHEALHNHYTQKSLSLSPGK
Bimagrumab Light Chain (LC)
(SEQ ID NO: 10)
QSALTQPASVSGSPGQSITISCTGTSSDVGSYNYVNWYQQHPGKAPK
LMIYGVSKRPSGVSNRFSGSKSGNTASLTISGLQAEDEADYYCGTFA
GGSYYGVFGGGTKLTVLGQPKAAPSVTLFPPSSEELQANKATLVCLI
SDFYPGAVTVAWKADSSPVKAGVETTTPSKQSNNKYAASSYLSLTPE
QWKSHRSYSCQVTHEGSTVEKTVAPTECS

In some embodiments, exemplary ActRII antibodies of the disclosure comprise a variable heavy chain comprising the CDR amino acid sequences of SEQ ID NO: 1 (CDRH1), SEQ ID NO: 2 (CDRH2), and SEQ ID NO: 3 (CDRH3).

In some embodiments, exemplary ActRII antibodies of the disclosure comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII antibodies of the disclosure comprise a variable heavy chain comprising the CDR amino acid sequences of SEQ ID NO: 1 (CDRH1), SEQ ID NO: 2 (CDRH2), and SEQ ID NO: 3 (CDRH3); and comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII antibodies of the disclosure comprise a variable heavy chain (VH) comprising the amino acid sequence of SEQ ID NO: 7, or an amino acid sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or a variable light chain (VL) comprising the amino acid sequence of SEQ ID NO: 8, or an amino acid sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, exemplary ActRII antibodies of the disclosure comprise a heavy chain (HC) comprising the amino acid sequence of SEQ ID NO: 9, or an amino acid sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and/or a light chain (LC) comprising the amino acid sequence of SEQ ID NO: 10, or an amino acid sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, an ActRII antibody binds to ActRIIB with a KD of 100 nM or less, 10 nM or less, 1 nM or less. Preferably, an ActRII antibody binds to ActRIIB with an affinity of 100 pM or less (e.g. 100 pM, 50 pM, 10 pM, 1 pM or less). In some embodiments, an ActRII antibody binds to ActRIIB with an affinity of between 10 and 20 pM.

In some embodiments, an ActRII antibody binds to ActRIIB with a 5-fold greater affinity than to ActRIIA, more preferably 10-fold, still more preferably 50-fold, still more preferably 100-fold. In some embodiments, an ActRII antibody binds to ActRIIA with an affinity of 100 pM or more (i.e., 250 pM, 500 pM, 1 nM, 5 nM or more).

In some embodiments, ActRII antibodies of the disclosure comprise one or more CDRs of SEQ ID NOS: 11-75, as provided in WO2017156488, the content of which is herein incorporated by reference in its entirety. It is noted that the CDR sequences of SEQ ID NOS: 11-75 include the combinations of 6 CDRs described in WO2017156488, in which the ActRII antibodies are any of the antibodies of the A-H lineages, e.g., as provided in Table 1 of WO2017156488.

In some embodiments, ActRII antibodies of the disclosure comprise one or more CDRs of SEQ ID NOS: 76-81, as provided in WO2012064771, the content of which is herein incorporated by reference in its entirety. It is noted that the 6 CDR sequences of SEQ ID NOS: 76-81 correspond to the CDR sequences of SEQ ID NOS: 4-9, respectively of WO2012064771, wherein the antibody is Ab-14E1.

In some embodiments, ActRII antibodies of the disclosure comprise one or more CDRs of SEQ ID NO: 82-5049, as provided in WO2018183376, the content of which is herein incorporated by reference in its entirety. It is noted that the CDR sequences of SEQ ID NOS: 82-5049 include the combinations of 6 CDRs described in WO2018183376, in which the antibodies are any of the ActRII antibodies of the A-H lineages, e.g., as provided in Tables 3A-3F, of WO2018183376.

### III. ActRII Antibody Fixed Unit Dose

The compositions and methods provided herein comprise an ActRII antibody fixed unit dose configured to be administered subcutaneously. In exemplary embodiments, an ActRII antibody fixed unit dose of the disclosure comprises about 25 mg to about 600 mg of ActRII antibody per fixed unit dose. In some exemplary embodiments, an ActRII antibody fixed unit dose of the disclosure comprises about 100 mg to about 400 mg of ActRII antibody per fixed unit dose. In some exemplary embodiments, an ActRII antibody fixed unit dose comprises about 300 mg of ActRII antibody per fixed unit dose. In some exemplary embodiments, an ActRII antibody fixed unit dose comprises about 150 mg of ActRII antibody per fixed unit dose.

In other embodiments, an ActRII antibody fixed unit dose of the disclosure comprises about 100 mg to about 400 mg of an ActRII antibody. In some embodiments, an ActRII antibody fixed unit dose of the disclosure comprises about 100 mg to about 400 mg of an ActRII antibody, administered weekly and subcutaneously.

In exemplary embodiments, an ActRII antibody fixed unit dose comprises about 300 mg of an ActRII antibody. In some exemplary embodiments, a composition comprises a fixed unit dose of about 300 mg of an ActRII antibody, administered weekly and subcutaneously.

In exemplary embodiments, an ActRII antibody fixed unit dose comprises about 150 mg of an ActRII antibody. In some exemplary embodiments, a composition comprises a fixed unit dose of about 150 mg of an ActRII antibody, administered weekly and subcutaneously.

In some embodiments, the ActRII antibody may be present in the ActRII antibody fixed unit dose at about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or at about 600 mg per fixed unit dose. In some exemplary embodiments the ActRII antibody is present at about 100 mg to about 400 mg per fixed unit dose. In exemplary embodiments, the ActRII antibody is present at about 300 mg per fixed unit dose. In other exemplary embodiments the ActRII antibody is present at about 150 mg per fixed unit dose.

In some embodiments, the ActRII antibody is present in the ActRII antibody fixed unit dose in a concentration of about 25 mg/ml, about 50 mg/ml, about 75 mg/ml, about 100 mg/ml, about 125 mg/ml, about 150 mg/ml, about 175 mg/ml, about 200 mg/ml, about 225 mg/ml, about 250 mg/ml, about 275 mg/ml, about 300 mg/ml, about 325 mg/ml, about 350 mg/ml, about 375 mg/ml, about 400 mg/ml, about 425 mg/ml, about 450 mg/ml, about 475 mg/ml, about 500 mg/ml, about 525 mg/ml, about 550 mg/ml, about 575 mg/ml, or at about 600 mg/ml. In some embodiments, the ActRII antibody is present in the ActRII antibody fixed unit dose in a concentration of about 150 mg/ml.

In some embodiments, the ActRII antibody fixed unit dose comprises a volume of about 0.25 ml, about 0.5 ml, about 0.75 ml, about 1.0 ml, about 1.24 ml, about 1.5 ml, about 1.75 ml, about 2.0 ml, about 2.25 ml, about 2.5 ml, about 2.75 ml, about 3.0 ml, about 3.25 ml, about 3.5 ml, about 3.75 ml, about 4.0 ml, about 4.25 ml, about 4.5 ml, about 4.75 ml, or about 5.0 ml. In some embodiments, the ActRII antibody fixed unit dose comprises a volume of about 1 ml. In some embodiments, the ActRII antibody fixed unit dose comprises a volume of about 2 ml. In some embodiments, the ActRII antibody fixed unit dose comprises any of the aforementioned concentrations in any of the aforementioned volumes.

In some embodiments, the ActRII antibody fixed unit dose is administered periodically to subjects with a body weight of 200 kg or less. In some embodiments, the ActRII antibody fixed unit dose is administered periodically to subjects with a body weight of 300 kg or less. In some embodiments the ActRII antibody fixed unit dose is administered periodically to subjects with a body weight of 200 kg or greater as two fixed unit doses. In some embodiments the ActRII antibody fixed unit dose is administered periodically to subjects with a body weight of 300 kg or greater as two fixed unit doses. In some embodiments, the number of ActRII antibody fixed unit doses administered periodically increases step-wise with a subject's body weight. In some embodiments, the number of fixed unit doses increases about every 50 kg, about every 75 kg, about every 100 kg, about every 150 kg, about every 200 kg, or about every 300 kg of a subject's body weight.

In some embodiments, the ActRII antibody fixed unit dose is administered periodically to a subject every day, about six times a week, about five times a week, about four times a week, about three times a week, about twice a week, about once every week, or about once every two weeks. In some embodiments, the ActRII antibody fixed unit dose is administered periodically to a subject about every three weeks, about every four weeks, about every five weeks, about every six weeks, about every seven weeks, or about every eight weeks. In exemplary embodiments, the ActRII antibody fixed unit dose is administered periodically to the subject about once every week.

In some embodiments, the ActRII antibody fixed unit dose is administered to the subject for about a month, for about two months, for about three months, for about six months, for about a year, for about two years, for about five years, or indefinitely.

### Loading Dose

In some embodiments, administration of an ActRII antibody loading dose precedes the administration of an ActRII fixed unit dose. Without being bound by theory or mechanism, it is thought that administration of a loading dose decreases the time required to achieve a desired serum concentration of the ActRII fixed unit dose treatment. Accordingly, in some embodiments, the inclusion of one or more loading doses are provided herein.

A loading dose may be administered by any route of administration, including but not limited to subcutaneous or intravenous administration, irrespective of the subsequent subcutaneous administration of the fixed unit dose.

In some embodiments, one or more loading doses of an ActRII antibody are administered to a subject in need thereof at day 0 or week 0, prior to the administration of the ActRII antibody fixed unit dose. In some embodiments, one or more loading doses of an ActRII antibody are provided 1 day, about 2 days, about 3 days, about 4 days, about 5 days, about 6 days, about 1 week, about 2 weeks, about 3 weeks, or about 4 weeks, before administration of an ActRII antibody fixed unit dose. In some embodiments, one or more loading doses of an ActRII antibody are administered the same day or within 24 hours of an ActRII antibody fixed unit dose. In some embodiments, one loading dose is administered. In some embodiments, two loading doses are administered. In some embodiments, three loading doses are administered.

In some embodiments, a loading dose may be administered intravenously in a concentration of about 3 mg/kg per the subject's body weight to about 50 mg/kg per the subject's body weight, and all amounts in between. In some embodiments, an ActRII antibody loading dose is administered intravenously at about 30 mg/kg per the subject's body weight. In some embodiments, an ActRII antibody loading dose is administered intravenously at about 10 mg/kg per the subject's body weight.

In some embodiments, the ActRII antibody loading dose is administered in a dose of about 150 mg to about 4500 mg. In some embodiments, the ActRII antibody loading dose is administered in a dose of about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 2500 mg, about 3000 mg, about 4000 mg, about 4500 mg, about 5000 mg, about 5500 mg, or about 6000 mg. In some embodiments, the ActRII antibody loading dose is administered in a dose of about 210 mg. In some embodiments, the ActRII antibody loading dose is administered in a dose of about 700 mg.

In some embodiments, an ActRII antibody loading dose is administered in a dose of about 25 mg to about 600 mg. In some embodiments, an ActRII antibody loading dose is administered in a dose of about 100 mg to about 400 mg. In some embodiments, an ActRII antibody loading dose is administered in a dose of about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, or at about 600 mg. In some embodiments, an ActRII antibody loading dose is administered subcutaneously in a fixed unit dose of about 300 mg. In some embodiments, an ActRII antibody loading dose is administered subcutaneously in a fixed unit dose of about 150 mg.

In some embodiments, an ActRII antibody loading dose is administered subcutaneously. In some embodiments, an ActRII antibody loading dose is administered intravenously.

In some embodiments, one or more ActRII antibody loading doses are administered intravenously prior to administration of an ActRII antibody fixed unit dose of the disclosure.

In some embodiments, one or more ActRII antibody loading doses are administered subcutaneously prior to administration of an ActRII antibody fixed unit dose of the disclosure.

### Devices

As described herein, the fixed unit doses of the disclosure are configured for subcutaneous administration. In some embodiments, such an ActRII antibody fixed unit dose is housed in an autoinjector or other mechanical injection device. In some embodiments, such an ActRII antibody fixed unit dose is administered via a needle or syringe.

In some embodiments, such an ActRII antibody fixed unit dose is administered via an autoinjector or other mechanical injection device to subjects with a range of body weights. In some embodiments, an ActRII antibody fixed unit dose is designed to be self-administered via an autoinjector or other mechanical injection device.

In some embodiments, an ActRII antibody fixed unit dose is designed to be administered via needle or syringe to subjects with a range of body weights. In some embodiments, an ActRII antibody fixed unit dose is designed to be self-administered via a needle or syringe.

In exemplary embodiments, a composition, comprising a fixed unit dose of about 300 mg of an ActRII antibody, is designed to be administered subcutaneously and weekly via an autoinjector or other mechanical injection device.

In exemplary embodiments, a composition, comprising a fixed unit dose of about 300 mg of an ActRII antibody, is designed to be administered subcutaneously and weekly via a needle or syringe.

In other exemplary embodiments, a composition, comprising a fixed unit dose of about 150 mg of an ActRII antibody, is designed to be administered subcutaneously and weekly via an autoinjector or other mechanical injection device.

In exemplary embodiments, a composition, comprising a fixed unit dose of about 150 mg of an ActRII antibody, is designed to be administered subcutaneously and weekly via a needle or syringe.

### Subjects and Treatment

Provided herein are methods of treating a disease in a subject in need thereof, comprising subcutaneously administering to the subject a fixed unit dose of an ActRII antibody, wherein the fixed unit dose is administered to subjects with a range of body weights. An ActRII antibody fixed unit dose as part of a treatment provided herein may be used to treat a variety of diseases, including but not limited to: metabolic diseases, muscle wasting diseases, age-related muscle degeneration, heart diseases, and liver diseases.

In exemplary embodiments, treatments provided herein comprising an ActRII antibody in a fixed unit dose of about 25 mg to about 600 mg are useful for the treatment of metabolic diseases, muscle wasting diseases, age-related muscle degeneration, heart diseases, and liver diseases. In some exemplary embodiments, treatments provided herein comprising an ActRII antibody in a fixed unit dose of about 100 mg to about 400 mg are useful for the treatment of metabolic diseases, muscle wasting diseases, age-related muscle degeneration, heart diseases, and liver diseases. In some exemplary embodiments, treatments provided herein comprising an ActRII antibody in a fixed unit dose of about 300 mg, are useful for the treatment of metabolic diseases, muscle wasting diseases, age-related muscle degeneration, heart diseases, and liver diseases. In other exemplary embodiments, treatments provided herein comprising an ActRII antibody in a fixed unit dose of about 150 mg, are useful for the treatment of metabolic diseases, muscle wasting diseases, age-related muscle degeneration, heart diseases, and liver diseases.

In some embodiments, treatments provided herein comprising ActRII antibody fixed unit doses may be used to treat heart diseases, including but not limited to heart failure with preserved ejection fraction, heart failure with reduced ejection fraction, and heart failure with moderately reduced ejection fraction.

In some embodiments, treatments provided herein comprising an ActRII antibody fixed unit doses may be used to treat liver diseases, including but not limited to non-alcoholic fatty liver disease, e.g., non-alcoholic steatohepatitis.

In some embodiments, treatments provided herein comprising ActRII antibody fixed unit doses may be used to treat metabolic diseases, including but not limited to: metabolic syndrome, prediabetes, insulin resistance, obesity, diabetes (Type I and II), anti-psychotic drug-associated obesity, glucocorticoid-induced obesity, hypothalamic obesity associated with craniopharyngioma, and complex and monogenetic disorders associated with obesity. The monogenetic disorders associated with obesity in humans, may include but are not limited to Bardet-Biedl syndrome, and a disorder arising from a mutation in one or more of the following genes ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B, or combinations thereof. A metabolic disorder may also be associated with a complex genetic disorder, e.g., Prader-Willi syndrome.

In exemplary embodiments, treatments provided herein comprising an ActRII antibody in a fixed unit dose of about 25 mg to about 600 mg are useful for the treatment of obesity. In exemplary embodiments, treatments provided herein comprising an ActRII antibody in a fixed unit dose of about 100 mg to about 400 mg are useful for the treatment of obesity. In some exemplary embodiments, treatments provided herein comprising an ActRII antibody in a fixed unit dose of about 300 mg are useful for the treatment of obesity. In other exemplary embodiments, treatments provided herein comprising an ActRII antibody in a fixed unit dose of about 150 mg are useful for the treatment of obesity.

In some embodiments, treatments comprising ActRII antibody fixed unit doses may be used to treat an obesity-related disorder, including but not limited to: glucose intolerance, prediabetes, Type II diabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

Treatments comprising ActRII antibody fixed unit doses may be used to treat a subject who is overweight. In some embodiments, a subject has a BMI of 30 or greater. In some embodiments, a subject has a BMI of 27 or greater. In some embodiments, a subject has a BMI of 27 or greater, and has an obesity related disorder.

Treatments comprising ActRII antibody fixed unit doses of the disclosure may be used to treat a subject lacking glycemic control.

Treatments may be administered to subjects of any age, including under 18 years old. In some embodiments, the subject may be over 18 years old, over 30 years old, or over 40 years old. In some embodiments, the subject may be over 50 years old, over 60 years old, or over 80 years old.

In some embodiments, treatments comprising an ActRII antibody fixed unit doses reduce fat mass in the subject. In some embodiments, an ActRII antibody fixed unit dose reduces fat mass in a subject by at least 5% (e.g., 5%-30%) over the treatment period.

In some embodiments, treatments comprising ActRII antibody fixed unit doses increase lean mass in the subject. In some embodiments, the fixed unit dose increases lean mass in a subject by at least 1% (e.g., 1%-10%) over the treatment period.

In some embodiments, treatments comprising ActRII antibody fixed unit doses reduce fat mass and increase lean mass in the subject. In some embodiments, the ActRII antibody fixed unit dose reduces fat mass in a subject by at least 5% (e.g., 5%-30%) and increases lean mass by at least 1% (e.g., 1%-10%) over the treatment period.

In some embodiments, treatments comprising ActRII antibody fixed unit doses reduce fat mass and maintain lean mass in the subject. In some embodiments, an ActRII antibody fixed unit dose reduces fat mass in a subject by at least 5% (e.g., 5%-30%) and maintains lean mass over the treatment period.

In some embodiments, treatments comprising ActRII fixed unit doses reduce body weight in the subject. In some embodiments, the ActRII antibody fixed unit dose reduces body weight in a subject by at least 5%, at least 10%, at least 20%, at least 30%, at least 40%, or at least 50% over the treatment period.

In some embodiments, treatments comprising ActRII fixed unit doses reduce central adiposity in the subject. In some embodiments, the ActRII antibody fixed unit dose reduces central adiposity in a subject by at least 2% (e.g., 2%-20%) over the treatment period.

In some embodiments, treatments comprising ActRII fixed unit doses reduce liver fat and/or non-liver visceral fat in the subject. In some embodiments, the ActRII antibody fixed unit dose reduces central adiposity in a subject by at least 2% (e.g., 2%-20%) over the treatment period as assessed by magnetic resonance imaging (MRI).

In some embodiments, fat mass in response to an ActRII fixed unit dose is measured with bioelectrical impedance analysis (BIA), dual X-ray absorptiometry (DXA), magnetic resonance imaging (MRI), and/or waist circumference.

In some embodiments, treatments comprising ActRII fixed unit doses improves glycemic control in the subject.

In some embodiments, glycemic control in response to an ActRII fixed unit dose is measured by glucose and insulin levels, and the HOMA2 model is applied (www.dtu.ox.ac.uk/homacalculator/).

In some embodiments, the “Ctrough” or minimum serum concentration of an ActRII antibody in a subject may be measured to determine the efficacy of a treatment. In some embodiments, the efficacy of the treatment is assessed as a serum concentration of about 500%, about 400%, about 300%, about 200%, about 100%, about 90%, about 80%, or about 75% of the desired “Ctrough”. In some embodiments, a desired “Ctrough” is about 1 μg/ml to about 10 μg/ml. In some embodiments, a desired “Ctrough” is about 3 μg/ml to about 30 μg/ml. In some embodiments, a desired “Ctrough” is about 2.5 μg/ml, about 5 μg/ml, about 10 μg/ml, about 15 μg/ml, about 20 μg/ml, about 25 μg/ml, about 30 μg/ml, or about 35 μg/ml.

In some embodiments, the efficacy of an ActRII fixed unit dose is determined by about 100%, about 90%, about 80%, about 75%, about 50%, or about 25% improvement in at least one of the following measurements: body weight; bioelectrical impedance analysis (BIA) measurement of lean mass and/or fat mass; waist to hip ratio; waist to height ratio; dual X-ray absorptiometry (DXA) measurement of lean mass and/or fat mass; magnetic resonance imaging (MRI) measurement of lean mass and/or fat mass; waist circumference; decreased BMI; blood lipids profile; leptin, lectin, adiponectin, and adipsin levels; IL-8 and/or IL-6 levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; the homeostasis model assessment 2 (HOMA2); and physical activity monitoring via actigraphy.

### Combination Therapies

In some embodiments, the ActRII antibody pharmaceutical compositions are administered as part of a combination therapy with an incretin agonist or another hormone agonist. The incretin agonist or other hormone agonist may be administered as part of the same or a separate formulation from the ActRII antibody, and may be administered at the same time or a different time than the ActRII antibody.

Incretin agonists suitable for a combination therapy with an ActRII antibody include but are not limited to: exenatide, exenatide extended-release, dulaglutide, liraglutide, lixisenatide, semaglutide, tirzepatide, cotadutide, noiiglutide, oxyntomodulin (e.g., mazdutide), retatrutide, albiglutide, beinaglutide, PEG-loxenatide, pemvidutide, and danuglipron (as described in WO 2023/028606, incorporated herein by reference in its entirety). Other hormone agonists suitable for combination therapy with an ActRII antibody include but are not limited to long-acting amylin receptor agonists, e.g., cagrilintide, dual amylin calcitonin receptor agonists (DACRAs), and Peptide YY agonists.

In some embodiments, the incretin agonist or other hormone agonist dose is administered weekly in a dose of about 0.25 mg from about week 0 to about week 4, in a dose of about 0.5 mg from about week 5 to about week 8, in a dose of about 1.0 mg from about week 8 to about week 12, in a dose of about 1.7 mg from about week 12 to about week 15, in a dose of about 2.4 mg from about week 16 to about week 20, and a dose of about 2.4 mg from about week 20 and thereafter. In some embodiments, the incretin agonist or other hormone agonist dose is administered weekly in a dose of about 5.0 mg, about 10 mg, or about 15 mg.

In some embodiments, the ActRII antibody and the incretin agonist or other hormone agonist are both administered subcutaneously, according to any of the above doses.

In some embodiments, the ActRII antibody is administered prior to the incretin agonist or other hormone agonist. In some embodiments, the ActRII antibody is administered at least 12 weeks prior, at least 10 weeks prior, at least 8 weeks prior, at least 6 weeks prior, at least 4 weeks prior, at least 2 weeks prior, at least 1 week prior, at least 1 day prior, or at least 1 hour prior to the administration of the incretin agonist or other hormone agonist.

In some embodiments, the incretin agonist or other hormone agonist is administered prior to the ActRII antibody. In some embodiments, the incretin agonist or other hormone agonist is administered at least 2 weeks prior, at least 1 week prior, at least 5 days prior, at least 4 days prior, at least 2 days prior, at least 1 day prior, at least 6 hours prior, or at least 1 hour prior to the ActRII antibody.

In some embodiments, the ActRII antibody and the incretin agonist or other hormone agonist are administered without regard for the order of administration.

In some embodiments, the ActRII antibody and the incretin agonist or other hormone agonist are administered at essentially the same time. In some embodiments, the ActRII antibody and the incretin agonist or other hormone agonist are administered in the same formulation.

### IV. Pharmaceutical Compositions

The compositions provided herein comprising ActRII antibody fixed unit doses are configured for subcutaneous administration. In exemplary embodiments, the compositions are formulated as pharmaceutical compositions including an ActRII antibody comprising the sequence of SEQ ID NOS: 1-6 and/or SEQ ID NOS: 7 and 8. In some exemplary embodiments, the compositions are formulated as pharmaceutical compositions including an ActRII antibody comprising the sequence of SEQ ID NO: 7 or a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto, and/or the sequence of SEQ ID NO: 8 or a sequence with at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments the pharmaceutical composition includes an excipient, or carrier, e.g., an aqueous carrier. A variety of aqueous carriers can be used, e.g., buffered saline. The pharmaceutical compositions may contain pharmaceutically acceptable auxiliary substances as those required to approximate physiological conditions such as pH and buffering agents, toxicity countering agents, e.g., disodium phosphate dihydrate, monosodium phosphate, sodium acetate, sodium chloride, potassium chloride, calcium chloride, hydrochloric acid, sodium hydroxide, L-histidine, L-histidine hydrochloride, and sodium lactate. The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)). The pharmaceutical compositions may contain pharmaceutically acceptable auxiliary substances such as those that contribute to the stability and activity of the pharmacologically active agent or agents, including but not limited to trehalose, sucrose, or other sugars and polysorbate 20, polysorbate 60, polysorbate 80 or other emulsifiers or stabilizers.

### Coformulations

In some embodiments, the ActRII antibody pharmaceutical compositions are co-formulated with an incretin agonist or another hormone agonist. Incretin agonists suitable for co-formulation include but are not limited to: exenatide, exenatide extended-release, dulaglutide, liraglutide, lixisenatide, semaglutide, tirzepatide, cotadutide, noiiglutide, oxyntomodulin (e.g., mazdutide), retatrutide, albiglutide, beinaglutide, PEG-loxenatide, pemvidutide, and danuglipron (as described in WO 2023/028606, incorporated herein by reference in its entirety). In some embodiments, the incretin agonist is present in the pharmaceutical composition with an ActRII antibody in a dose of about 0.005 mg to about 3.0 mg. In some embodiments, the incretin agonist is present in the pharmaceutical composition with an ActRII antibody in a dose of about 0.05 mg to about 2.0 mg. In some embodiments, the incretin agonist is present in the pharmaceutical composition with an ActRII antibody in a dose of about 0.5 mg to about 1.0 mg. In some embodiments, the concentration of the incretin agonist may vary or increase incrementally during the treatment.

In some embodiments, an incretin agonist is present in the pharmaceutical composition in a dose of about 0.005 mg to about 3.0 mg and the ActRII antibody is present in a dose of about 25 mg to about 600 mg. In some embodiments, an incretin agonist is present in the pharmaceutical composition in a dose of about 0.005 mg to about 3.0 mg and the ActRII antibody is present in a dose of about 100 mg to about 400 mg. In some embodiments, an incretin agonist is present in the pharmaceutical composition in a dose of about 0.005 mg to about 3.0 mg and the ActRII antibody is present in a dose of about 300 mg. In some embodiments, an incretin agonist is present in the pharmaceutical composition in a dose of about 0.005 mg to about 3.0 mg and the ActRII antibody is present in a dose of about 150 mg.

Other hormone agonists suitable for co-formulation with an ActRII antibody in the pharmaceutical composition include but are not limited to long-acting amylin receptor agonists, e.g., cagrilintide, dual amylin calcitonin receptor agonists (DACRAs), and Peptide YY agonists.

### EXAMPLES

### Example 1: Methods of Clinical Study to Evaluate Subcutaneous Dosing of an ActRII Antibody

The purpose of this study was to assess the pharmacokinetic (PK) and pharmacodynamic (PD) effects of subcutaneous (s.c.) dosing of bimagrumab, an ActRII antibody. The pharmacodynamic effects of interest in this study were fat mass and lean mass.

### Primary Objectives

1. To evaluate the safety and tolerability of different s.c. dosing of bimagrumab.
2. To determine the local tolerability of s.c. administration of bimagrumab
3. To evaluate the pharmacokinetics of different s.c. relative to intravenous (i.v.) dosing of bimagrumab

### Endpoints for Primary Objectives

1. Physical exam/body weight
2. Vital signs
3. ECG
4. Safety laboratories
5. Adverse events (AEs)/Serious adverse events SAEs)
6. Pain assessment by visual analog scale (VAS)
7. Investigator's assessment for tenderness, erythema/redness, induration/swelling
8. PK (sampling of full profiles after first and last dose and pre-dose during treatment)
9. Basic PK parameters: Cmax, Tmax, Cmin, AUCtau, AUClast (after the last dose only), Race

### Secondary Objective

To determine the absolute bioavailability of bimagrumab given by s.c. administration.

### Endpoints for Secondary Objective

The absolute bioavailability was determined by modeling, as bimagrumab exhibited target mediated drug disposition (TMDD), in which a drug binds with such high affinity to its pharmacological target that this affects its pharmacokinetic characteristics.

### Exploratory Objective

To explore the pharmacodynamics and the PK/PD relationship of different s.c. and i.v. dose regimens of bimagrumab.

### Endpoints for Exploratory Objective

Pharmacokinetics (PK): full PK profiles after first and last dose and pre-dose during treatment and trough samples before each dose. Pharmacodynamics: lean body mass (LBM), including appendicular LBM, by dual energy X-ray absorptiometry (DXA) before and during the treatment and at the end of follow-up period.

### Study Design

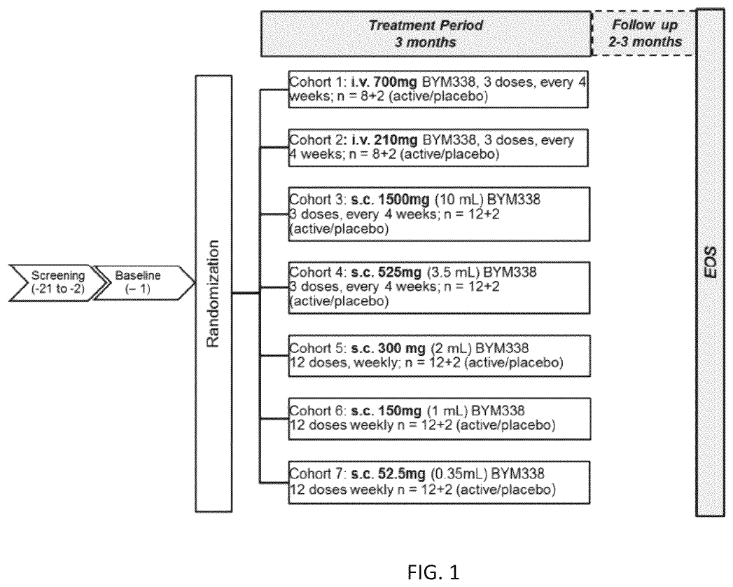

This was an exploratory, randomized, placebo-controlled, multiple-dose study in elderly healthy male and female subjects age 70 years and older. It was designed with a total of 7 treatment groups who received either active or placebo in a parallel group-comparison design, see FIG. 1.

This study was designed to combine the evaluation of safety/tolerability and the pharmacokinetic and pharmacodynamic characteristics of multiple doses of bimagrumab given as different s.c. or i.v. regimens every 4 weeks, as well as more frequent weekly s.c. dosing regimens. The s.c. dosing every 4 weeks was administered with an infusion pump.

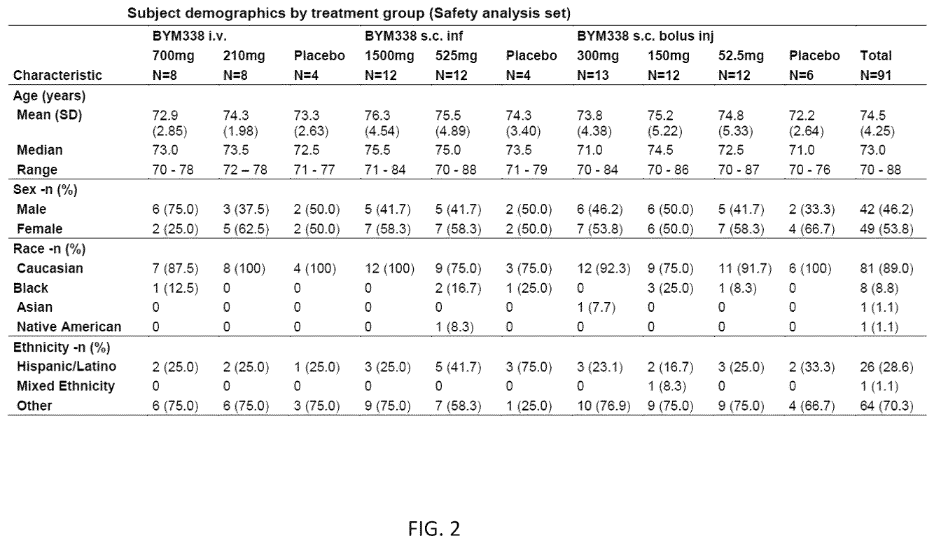

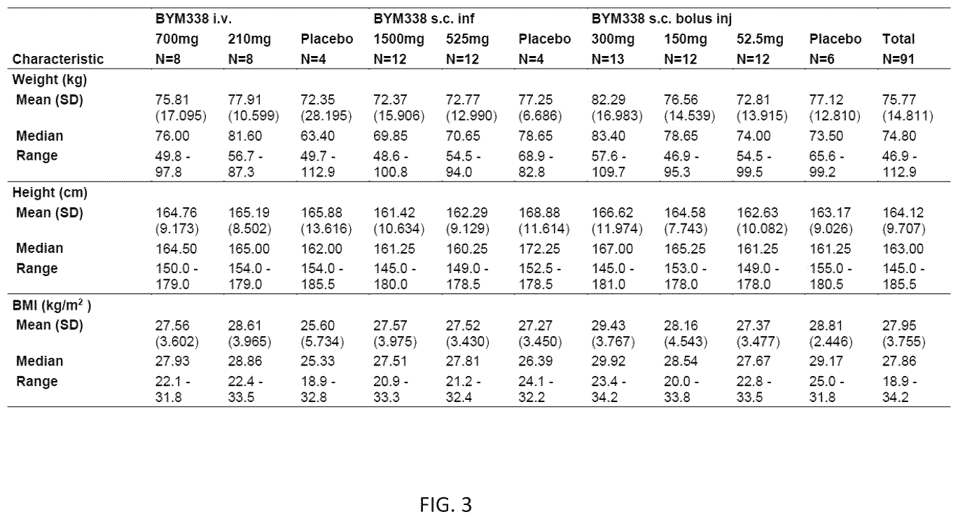

A total of 91 male or female elderly subjects with stable health and medication use were enrolled in the study and randomized. Overall, demographic characteristics were generally balanced between different dose groups (see FIGS. 2 and 3). The mean (SD) age of the subjects was 74.5 (4.25) years, height was 164.12 (9.70) cm, weight was 75.77 (14.81) kg and BMI was 27.95 (3.75) kg/m². The subject population involved was predominantly Caucasian (89%), with a high proportion of Hispanic/Latino (28.6%). Of the 91 subjects enrolled, 49 were female.

### Treatment Arms

Subjects were assigned to one of the following treatment arms:
Intravenous infusion (cohorts 1 & 2) 8 active+2 placebo per cohort; n=20:
   700 mg by i.v. infusion over 30 min, given every 4 weeks at Week 0, 4, 8. Total dose given over the study: 700 mg×3=2100 mg
   210 mg by i.v. infusion over 30 min, given every 4 weeks at Week 0, 4, 8. Total dose given over the study: 210 mg×3=630 mg
High and mid volume s.c. (cohorts 3 & 4) 12 active+2 placebo each per cohort; n=28:
   1500 mg (10 mL) by s.c. infusion over 30 min, given every 4 weeks at Week 0, 4, 8. Total dose given over the study: 1500 mg×3=4500 mg
   525 mg (3.5 mL) by s.c. infusion over 10 min, given every 4 weeks at Week 0, 4, 8. Total dose given over the study: 525 mg×3=1575 mg
Low volume s.c. (cohorts 5, 6 & 7) 12 active+2 placebo per cohort; n=43:
   300 mg (2 mL) by s.c. injection (bolus), given every week up to Week 11. Total dose given over the study: 300×12=3600 mg
   150 mg (1 mL) by s.c. injection (bolus), given every week up to Week 11. Total dose given over the study: 150×12=1800 mg
   52.5 mg (0.35 mL) by s.c. injection (bolus), given every week up to Week 11. Total dose given over the study: 52.5×12=630 mg

### Pharmacodynamic Assessments

Dual energy X-ray absorptiometry (DXA) was used to assess changes in body composition, including total lean body mass (LBM) and fat body mass (FBM) and appendicular skeletal mass index (ASMI). DXA instruments use an X-ray source that generates and is split into two wavelengths to measure bone mineral mass and soft tissue, from which fat and fat-free mass (or lean body mass) were estimated.

### Safety Assessments

Safety assessments consisted of collecting all adverse events (AEs) and serious adverse events (SAEs). The regular monitoring of hematology, blood chemistry and urine was performed at the study center, and regular assessments were done of vital signs, physical condition and body weight and ECG. Local tolerability assessment was also performed and immunogenicity (IG) samples were collected.

### Pharmacokinetic Assessments

Pharmacokinetic (PK) samples were obtained and evaluated in all subjects at all dose levels using a validated bridging enzyme-linked immunosorbent assay (ELISA) method, the anticipated Lower Limit of Quantification (LLOQ) was 0.176 μg/mL. The linear trapezoidal rule was used for AUC calculation. As bimagrumab exhibits a non-linear PK profile, also known as TMDD, AUC_{inf}, half-life (T½) and Clearance (CL) and Volume of distribution (V) were not derived using noncompartmental analysis (NCA). The accumulation ratio (R_{acc}) was calculated using AUCₜₐᵤ values obtained from a dosing interval after the first and last dose as [(AUCₜₐᵤ, last dose)/(AUCₜₐᵤ, first dose)]. As bimagrumab exhibits non-linear PK profile, with CL dependent on bimagrumab concentrations, the AUC cannot be used to infer absolute bioavailability and dose proportionality. Therefore, a model-based approach was used to characterize the subcutaneous bioavailability

### Example 2: Pharmacokinetic Results of Clinical Study to Evaluate Subcutaneous Dosing of an ActRII Antibody

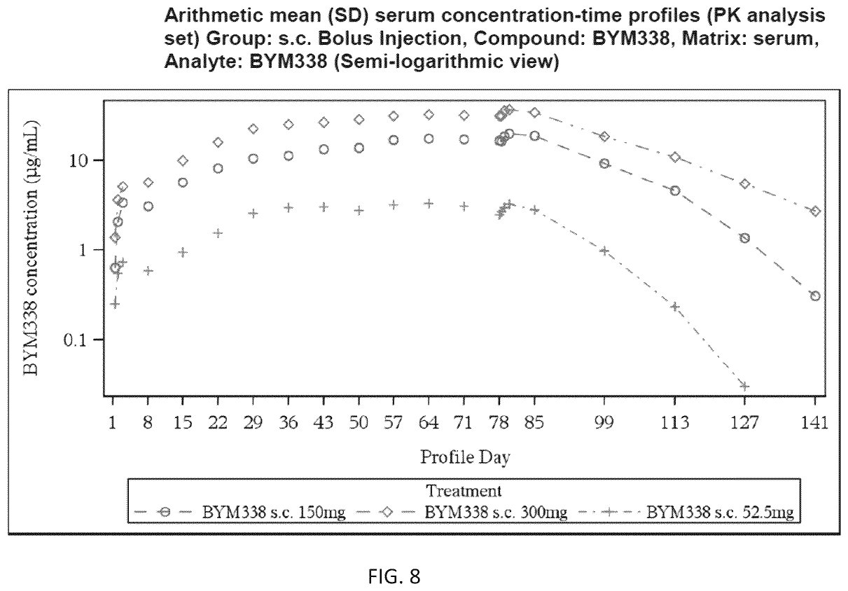

Notably, the 300 mg subcutaneous bolus dose exhibited no detectable TMDD effect, indicating that for this method of administration, a 300 mg dose was high enough to maintain saturation of the clearance. As demonstrated in FIG. 8, a 300 mg subcutaneous (s.c.) bolus dose of an ActRII antibody is predicted to maintain serum concentration above 10 μg/ml at a relatively steady level from about day 15 to about day 85. Similarly, a 150 mg subcutaneous bolus dose of an ActRII antibody is predicted to maintain serum concentration at about 10 μg/ml from about day 29 to about day 85.

### Intravenous

Cohorts 1 and 2 (i.v. infusion): As expected, a TMDD was observed on the concentration-time profiles. The variability was moderate with around 30-40% coefficient of variability (CV; range 17 to 47% CV). The accumulation ratio, based on AUCtau, was relatively low (range of 1.15 to 1.37), but it could be observed from the Cmin that the steady state might not have been reached with three monthly administrations. As expected with i.v. administration, the peak-to-trough ratio was relatively large.

### Subcutaneous

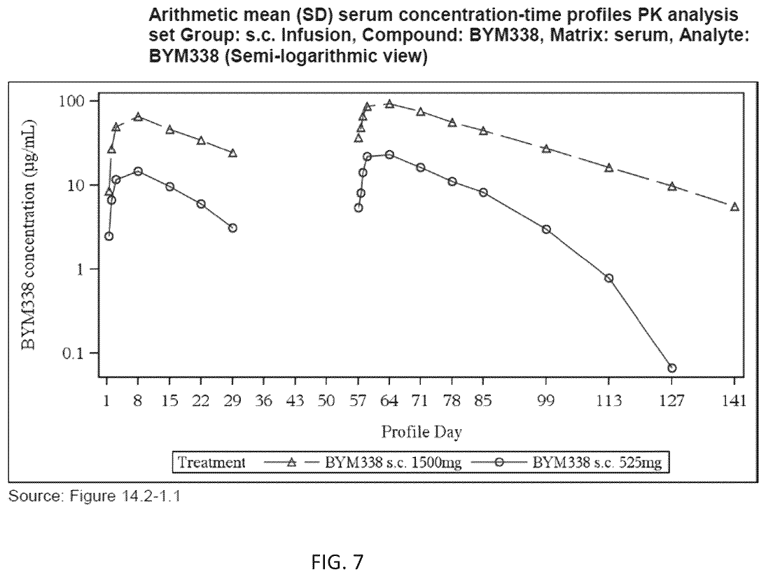

Cohorts 3 and 4 (s.c. infusion): The absorption of bimagrumab following s.c. infusion was relatively slow (Tmax around one week post-dose). The TMDD profile was observed only at the lower dose (525 mg) (FIG. 7). The variability was moderate (with a range of 29 to 39% CV). The accumulation ratio, based on AUCtau, was slightly higher than with i.v. infusion (between 1.72 and 1.92), but it could be also observed from the Cmin that the steady state might not have been reached with three monthly administrations. The peak-to-trough ratio was moderate.

Cohorts 5, 6 and 7 (s.c. bolus): The absorption of bimagrumab following the s.c. bolus was faster at low doses following the first dose (FIG. 8). The concentration-time profiles were relatively flat after the final dose with a low peak-to-trough ratio. The variability of PK parameters was relatively high with a CV around 50% for 300 mg and 150 mg and around 100% for 52.5 mg. The accumulation ratio, based on AUCtau, was much higher than in the i.v. and s.c. infusion groups. For most of the subjects, the steady state, based on Cmin, was reached after 9 weekly doses (Day 57). Notably, the TMDD profile could be observed at the low doses (i.e. 52.5 mg and 150 mg) but not at 300 mg, demonstrating that for a 300 mg subcutaneous bolus, the concentration was high enough to maintain saturation of the clearance.

### Example 3: Pharmacodynamic Results of Clinical Study to Evaluate Subcutaneous Dosing of an ActRII Antibody

Administration via intravenous and subcutaneous (both bolus and infusion) showed similar effects on lean body mass increase and fat body mass decrease.

### LBM and Appendicular LBM

A dose-dependent plateau effect with respect to lean mass increase, of the order of 1.5-2 kg (i.e. an increase of ˜4-6% from baseline) at maximal doses was observed at the end of each treatment period (Day 85), whether bimagrumab was administered i.v. or s.c. (bolus and infusion).

### FBM

A dose-dependent linear decrease in whole-body fat mass, up to ˜2 to 3 kg at maximal doses, was observed at the end of each treatment period (Day 85), whether bimagrumab was administered i.v. or s.c. (bolus and infusion). For the highest doses, during the follow-up treatment period, this loss of fat further increased to ˜3 to 4 kg in the i.v. and s.c. infusion groups and was maintained in the highest dose s.c. infusion group.

### Example 4: Model Predictions of Subcutaneous Dosing of an ActRII Antibody

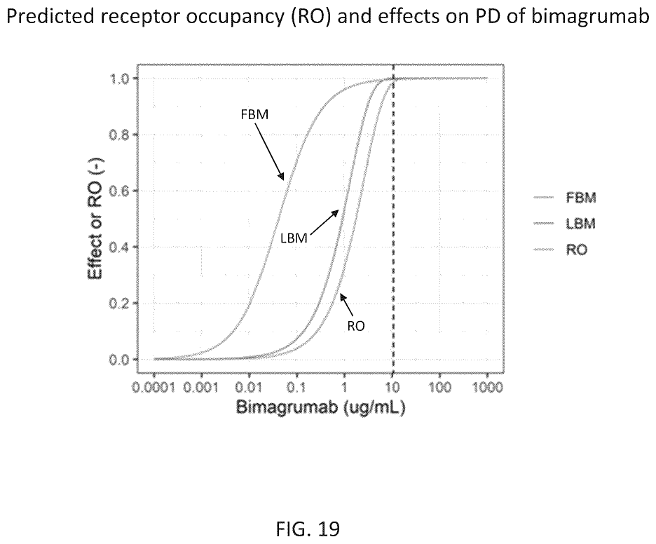

Computational models of the ActRII antibody bimagrumab effects on PK and PD were developed based on the above clinical studies (Examples 1-3). The model predicts full ActRII receptor occupancy (RO) (FIG. 19) with an ActRII serum concentration of about 10 μg/ml for most subjects.

The effects of loading doses were modeled in order to predict the number of subjects across a range of body weights with an ActRII serum concentration above the RO of 10 μg/ml, and to predict the time required to reach this concentration.

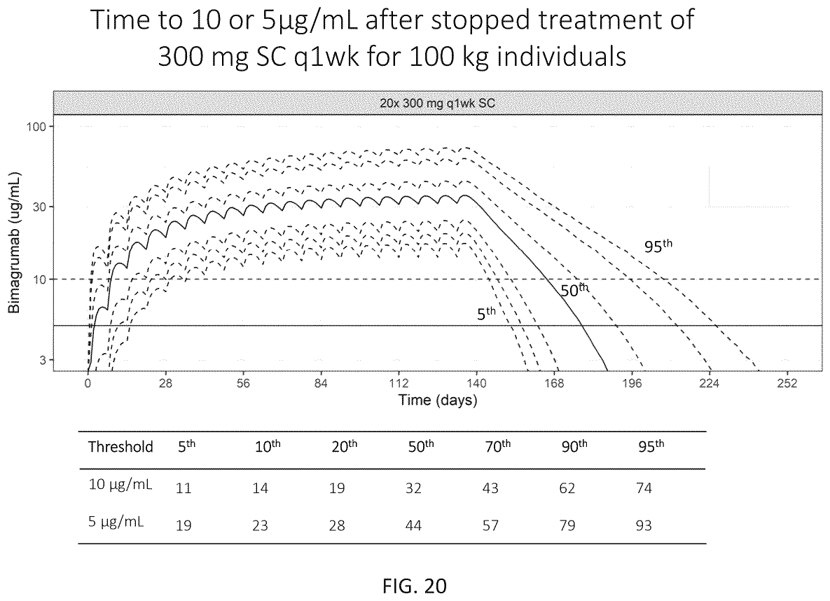

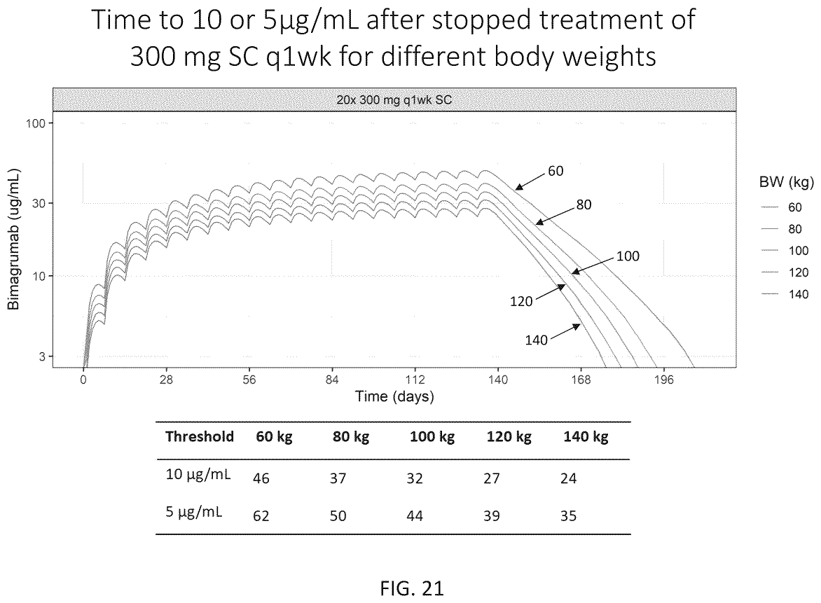

FIG. 20 shows the predicted time to reach various bimagrumab concentrations in a population of 2500 subjects, each weighing 100 kg, in which 300 mg bimagrumab is administered weekly and subcutaneously without a loading dose. The threshold levels of 5 μg/ml and 10 μg/ml are indicated in the graph and in the table below. FIG. 21 shows the predicted time to reach various bimagrumab concentrations in a population of 2500 subjects, of weights from 60 to 140 kg.

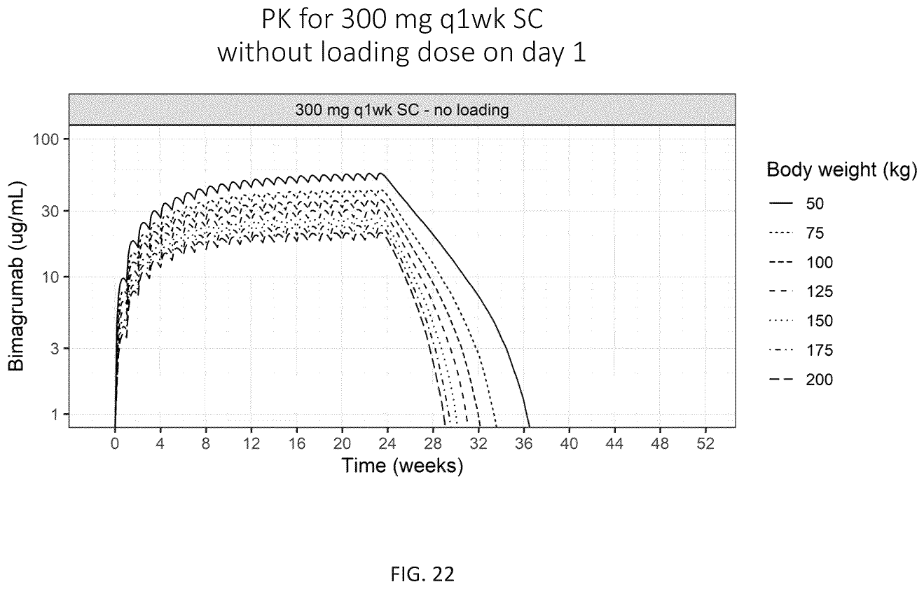

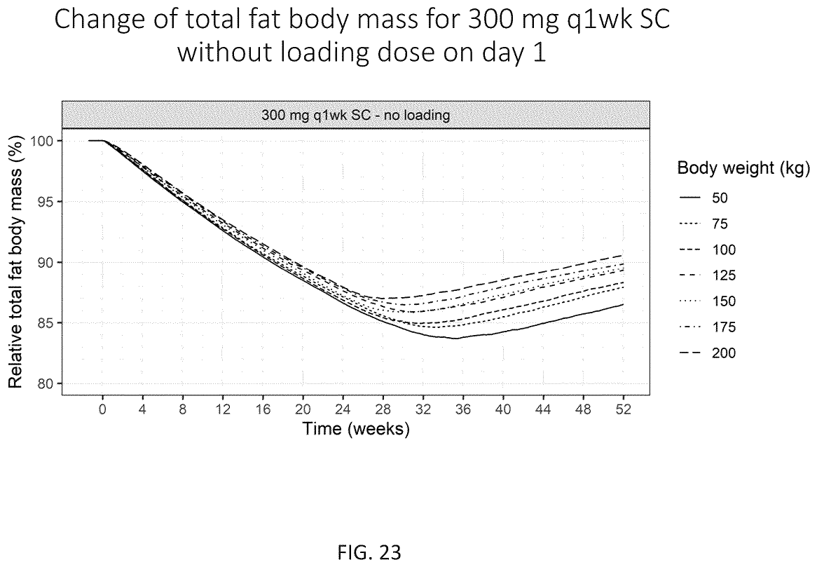

FIG. 22 shows the predicted bimagrumab concentration over time in a population of subjects, of weights from 50 to 200 kg, in which 300 mg bimagrumab is administered weekly and subcutaneously without a loading dose on day 1. FIG. 23 shows the predicted timing of change in total fat body mass in a population of subjects, of weights from 50 to 200 kg, in which 300 mg bimagrumab is administered weekly and subcutaneously without a loading dose on day 1.

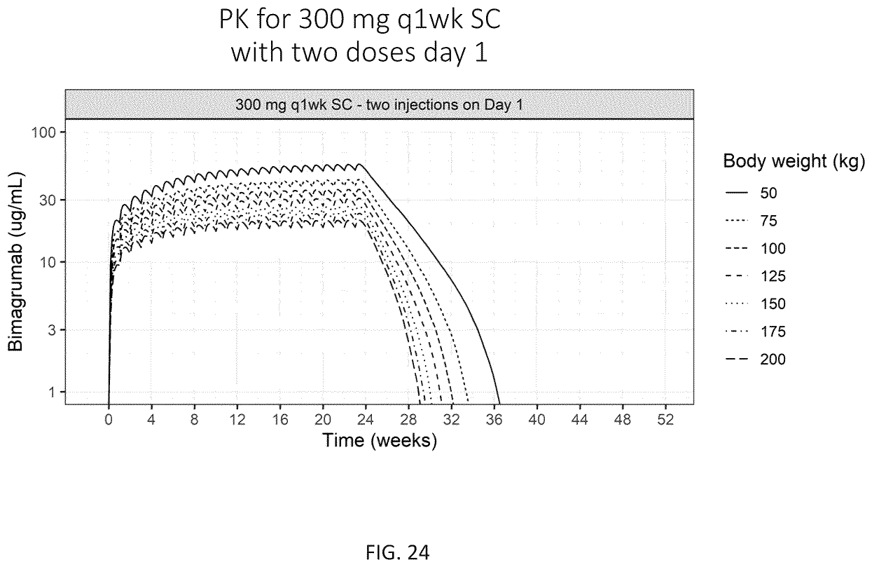

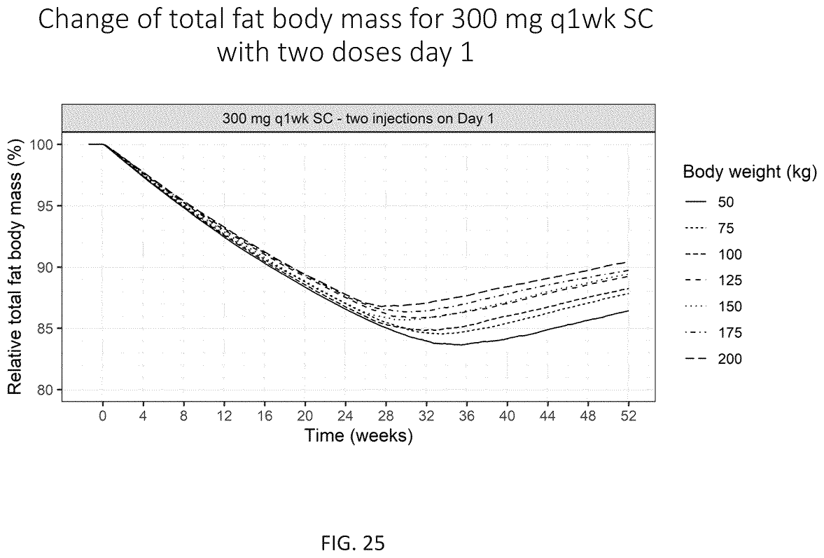

FIG. 24 shows the predicted bimagrumab concentration over time in a population of subjects, of weights from 50 to 200 kg, in which 300 mg bimagrumab is administered weekly and subcutaneously with two doses (i.e., with one loading dose) of 300 mg bimagrumab administered subcutaneously on day 1. Notably, the time to reach a median serum concentration of 10 μg/ml for all subjects was decreased with a loading dose on day 1. FIG. 25 shows the timing of change in total fat body mass in a population of subjects, of weights from 50 to 200 kg, in which 300 mg bimagrumab is administered weekly and subcutaneously with two doses (i.e., with one loading dose) of 300 mg bimagrumab administered subcutaneously on day 1.

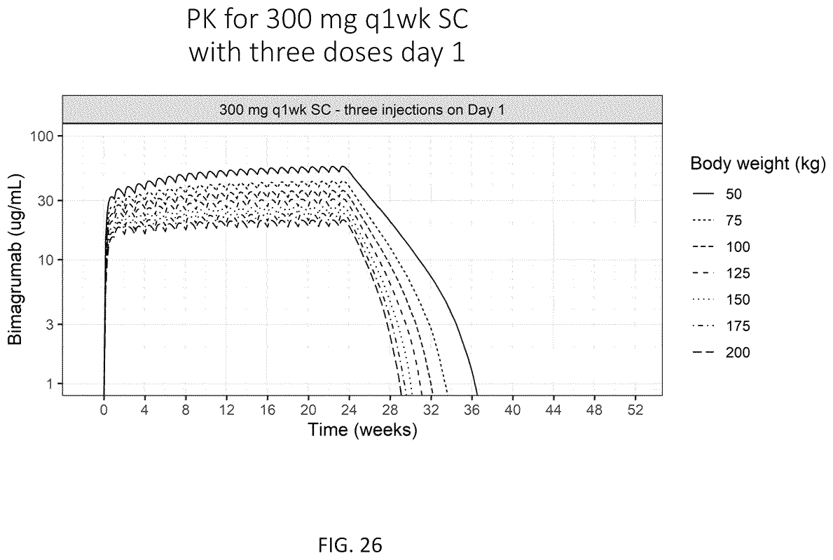

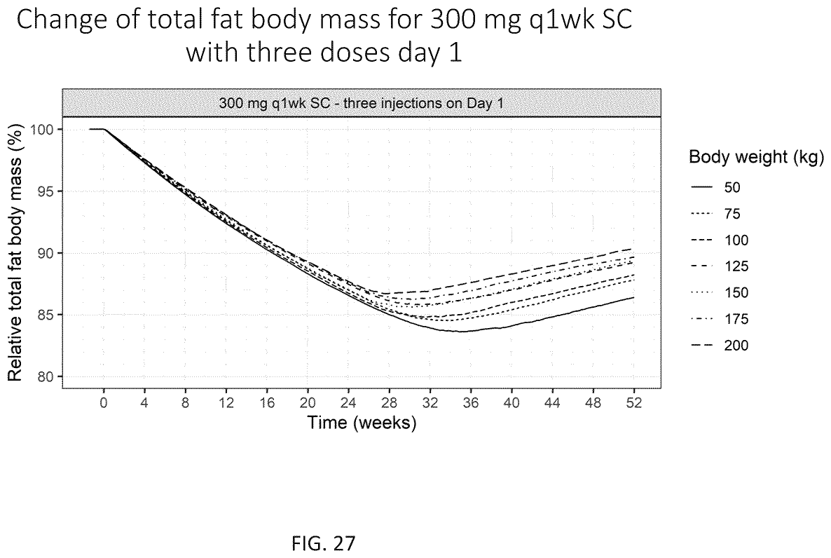

FIG. 26 shows the bimagrumab concentration over time in a population of subjects, of weights from 50 to 200 kg, in which 300 mg bimagrumab is administered weekly and subcutaneously with three doses (i.e., with two loading doses) of 300 mg bimagrumab administered subcutaneously on day 1. FIG. 27 shows the timing of change in total fat body mass in a population of subjects, of weights from 50 to 200 kg, in which 300 mg bimagrumab is administered weekly and subcutaneously with three doses (i.e., with two loading doses) of 300 mg bimagrumab administered subcutaneously on day 1.

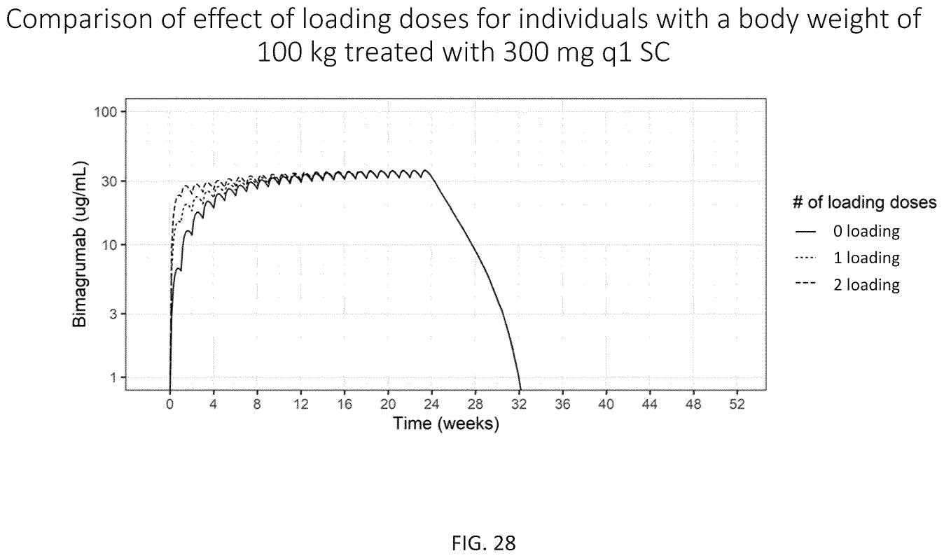

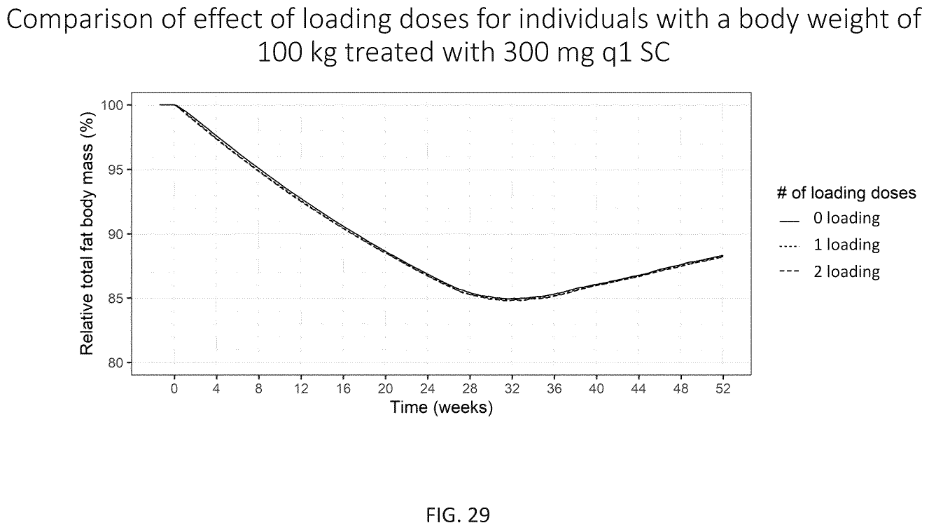

FIG. 28 shows a comparison of the effect of loading doses (0, 1, or 2 loading doses) on bimagrumab exposure in a population of subjects weighing 100 kg in which in which 300 mg bimagrumab is administered weekly and subcutaneously. FIG. 29 shows a comparison of the effect of loading doses (0, 1, or 2 loading doses) for a population of subjects with a weight of 100 kg in which in which 300 mg bimagrumab is administered weekly and subcutaneously.

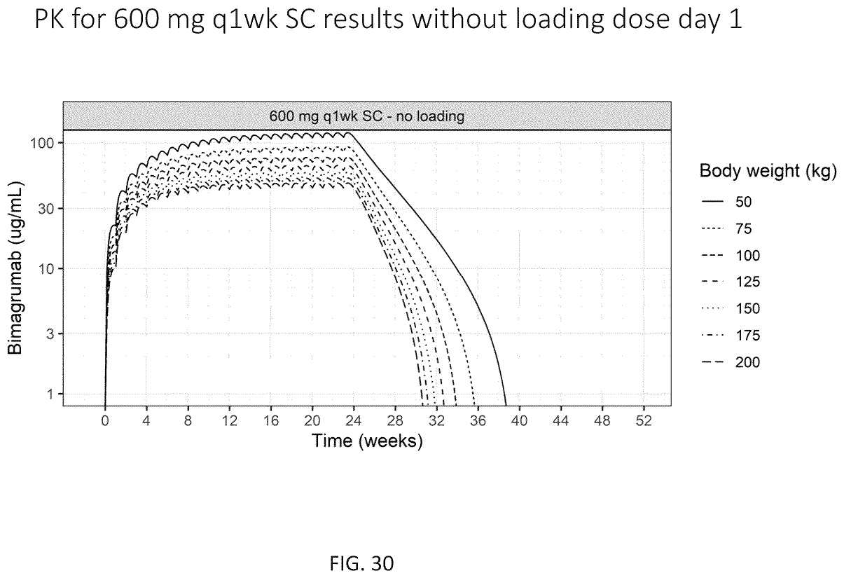

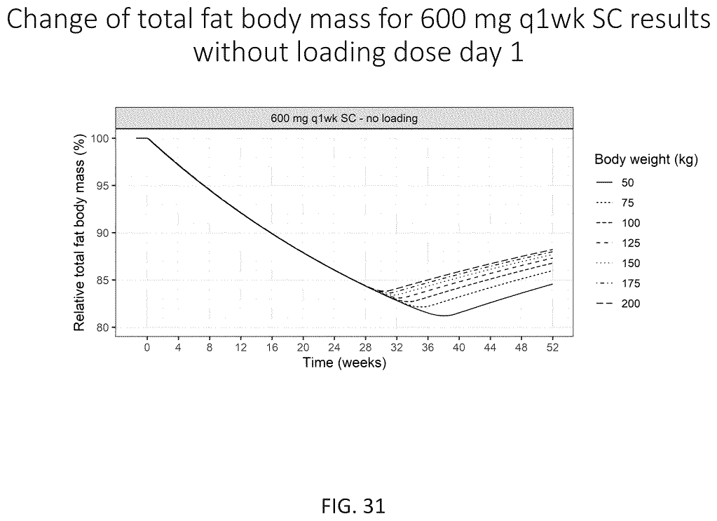

FIG. 30 and FIG. 31 show that increasing bimagrumab dose to 600 mg has little effect on the timing of exposure.

### Population Modeling Including Individual Variability

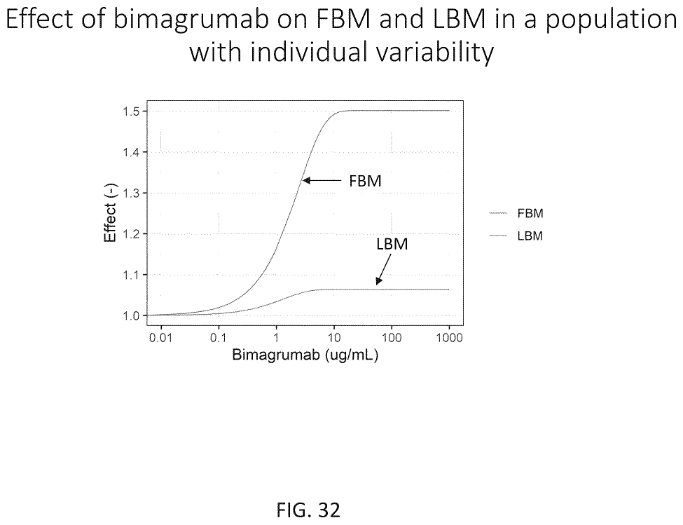

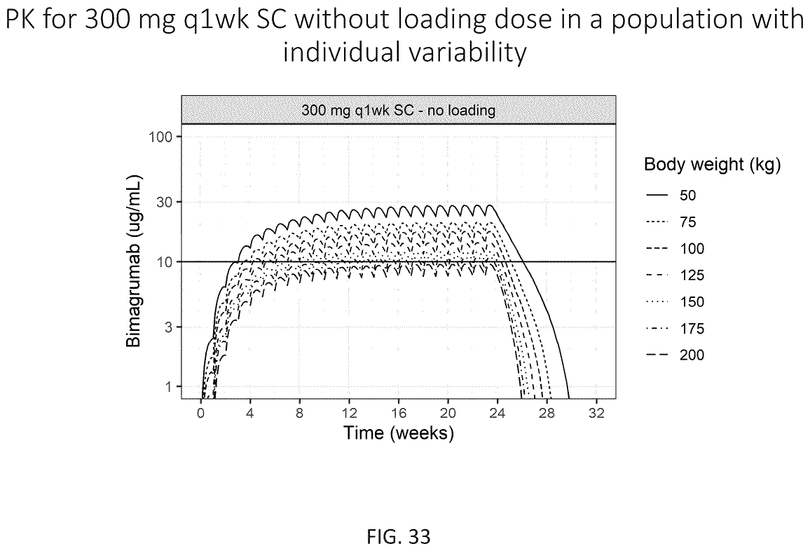

The models described herein predict that 300 mg administered weekly and subcutaneously, without a loading dose, is sufficient to reach a serum concentration of at least 10 μg/ml for most subjects. However, when individual response variability within the population is taken into account, as shown in FIG. 32, the concentration of bimagrumab required to have the maximal effect on fat body mass and lean body mass in a population increases to about 10-30 μg/ml. As shown in FIG. 33, in a model in which individual response variability within the population is taken into account, subjects with higher body weights do not reach 10 μg/ml with 300 mg administered weekly and subcutaneously, and a loading dose is required.

1.-78. (canceled)

79. A pharmaceutical composition comprising an ActRII antibody, wherein the ActRII antibody is present at about 25 mg to about 600 mg per fixed unit dose, and wherein the composition is configured for subcutaneous administration.

80. The pharmaceutical composition of claim 79, wherein the ActRII antibody is present at about 100 to about 400 mg per fixed unit dose.

81. The pharmaceutical composition of claim 80, wherein the ActRII antibody is present at about 150 mg per fixed unit dose.

82. The pharmaceutical composition of claim 80, wherein the ActRII antibody is present at about 300 mg per fixed unit dose.

83. The pharmaceutical composition of claim 79, wherein the ActRII antibody comprises the CDR amino acid sequences of SEQ ID NOS: 1-6.

84. The pharmaceutical composition of claim 83, wherein the ActRII antibody comprises: the VH amino acid sequence of SEQ ID NO: 7, or a sequence with at least 80% sequence identity thereto, and the VL amino acid sequence of SEQ ID NO: 8, or a sequence with at least 80% sequence identity thereto.

85. The pharmaceutical composition of claim 84, wherein the ActRII antibody comprises: the amino acid sequence of SEQ ID NO: 9, or a sequence with at least 80% sequence identity thereto, and the amino acid sequence of SEQ ID NO: 10, or a sequence with at least 80% sequence identity thereto.

86. The pharmaceutical composition of claim 85, wherein the pharmaceutical composition comprises an incretin agonist.

87. The pharmaceutical composition of claim 86, wherein the incretin agonist is selected from the group consisting of exenatide, exenatide extended-release, dulaglutide, liraglutide, lixisenatide, semaglutide, tirzepatide, cotadutide, noiiglutide, oxyntomodulin, retatrutide, albiglutide, beinaglutide, PEG-loxenatide, pemvidutide, and danuglipron.

88. A method of treating a metabolic disease in a subject in need thereof, comprising subcutaneously administering to the subject at about 25 mg to about 600 mg fixed unit dose of an ActRII antibody.

89. The method of claim 88, wherein the ActRII antibody is present in at about 100 mg to about 400 mg per fixed unit dose.

90. The method of claim 89, wherein the ActRII antibody is present at about 150 mg per fixed unit dose.

91. The method of claim 89, wherein the ActRII antibody is present at about 300 mg per fixed unit dose.

92. The method of claim 88, wherein the disease is a metabolic disease is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

93. The method of claim 88, wherein the disease is a muscle wasting disease or sarcopenia.

94. The method of claim 88, wherein the subject has an obesity disorder.

95. The method of claim 88, wherein the ActRII antibody comprises the CDR amino acid sequences of SEQ ID NOS: 1-6.

96. The method of claim 88, wherein the ActRII antibody comprises: the VH amino acid sequence of SEQ ID NO: 7, or a sequence with at least 80% sequence identity thereto, and the VL amino acid sequence of SEQ ID NO: 8, or a sequence with at least 80% sequence identity thereto.

97. The method of claim 96, wherein the ActRII antibody comprises: the HC amino acid sequence of SEQ ID NO: 9, or a sequence with at least 80% sequence identity thereto, and the LC amino acid sequence of SEQ ID NO: 10, or a sequence with at least 80% sequence identity thereto.

98. The method of claim 97, wherein the fixed unit dose of an ActRII antibody is administered as a part of a combination therapy with an incretin agonist.

99. The method of claim 98, wherein the incretin agonist is selected from exenatide, exenatide extended-release, dulaglutide, liraglutide, lixisenatide, semaglutide, tirzepatide, cotadutide, noiiglutide, oxyntomodulin, retatrutide, albiglutide, beinaglutide, PEG-loxenatide, pemvidutide, and danuglipron.

100. The method of claim 98, wherein the ActRII antibody is co-formulated with an incretin agonist.
